(19) United States
(12) Patent Application Publication
(10) Pub. No.: US 2025/0099245 A1
(43) Pub. Date: Mar. 27, 2025

(54) CONTROLLED DEPLOYMENT, RECAPTURE, AND BAIL OUT OF TAVR VALVE

(71) Applicant: BOSTON SCIENTIFIC SCIMED, INC., MAPLE GROVE, MN (US)

(72) Inventors: Levi Joel Wolterstorff, Saint Paul, MN (US); Chenhao Fu, Plymouth, MN (US); Joshua Mark Inouye, Brooklyn Park, MN (US); John M. Edgell, Plymouth, MN (US); Kelsey Rae Cooper, Andover, MN (US); Philip Andrew Litecky, Forest Lake, MN (US)

(73) Assignee: BOSTON SCIENTIFIC SCIMED, INC., MAPLE GROVE, MN (US)

(21) Appl. No.: 18/895,991

(22) Filed: Sep. 25, 2024

Related U.S. Application Data
(60) Provisional application No. 63/585,257, filed on Sep. 26, 2023.

(51) Int. Cl. A61F 2/24; A61F 2/00; A61F 2/95; A61F 2/966

(52) U.S. Cl. CPC A61F 2/2439; A61F 2/2418; A61F 2/2436; A61F 2002/0081; A61F 2002/9511; A61F 2002/9534; A61F 2002/9665

(57) ABSTRACT

A delivery system for delivering, adjusting, and/or recapturing a stent-valve includes an inner shaft and an expandable stent-valve coupled to the inner shaft. The stent-valve has an upper portion, a lower portion, and a valve. The delivery system also includes a distal sheath disposed over at least the lower portion, a proximal sheath disposed over at least the upper portion, and at least one cinching member coupled to the stent-valve and extending through one of the inner shaft, the distal sheath, or the proximal sheath. The upper and lower portions of the stent-valve are configured to move to an expanded configuration when the proximal and distal sheaths are withdrawn, respectively. After at least a portion of the stent-valve has been moved to the expanded configuration, the at least one cinching member is configured to at least partially radially compress at least a portion of the stent-valve.

### CROSS-REFERENCE TO RELATED APPLICATIONS

This application claims the benefit of priority of U.S. Provisional Application No. 63/585,257 filed Sep. 26, 2023, the entire disclosure of which is hereby incorporated by reference.

### TECHNICAL FIELD

The disclosure pertains to medical devices and more particularly to delivery systems for replacement heart valves, and methods for using such medical devices and systems.

### BACKGROUND

A wide variety of medical devices have been developed for medical use including, for example, medical devices utilized to replace heart valves. Heart function can be significantly impaired when a heart valve is not functioning properly. When the heart valve is unable to close properly, the blood within a heart chamber can regurgitate, or leak backwards through the valve. Valve regurgitation may be treated by replacing or repairing a diseased valve, such as an aortic valve. Surgical valve replacement is one method for treating the diseased valve, however this requires invasive surgical openings into the chest cavity and arresting of the patient's heart and cardiopulmonary bypass. Minimally invasive methods of treatment, such as transcatheter aortic valve implantation (TAVI) or transcatheter aortic valve replacement (TAVR), generally involve the use of delivery catheters that are delivered through arterial passageways or other anatomical routes into the heart to replace the diseased valve with an implantable prosthetic heart valve.

In some cases, readjustment, recapture, or bail out with the replacement heart valve during implantation is desired. Of the known delivery systems and methods for implanting a prosthetic heart valve, each has certain advantages and disadvantages. There is an ongoing need to provide alternative delivery systems as well as alternative methods for manufacturing and using the medical devices.

### SUMMARY

This disclosure provides design, material, manufacturing method, and use alternatives for medical devices. An example delivery system configured to deliver, adjust, and/or recapture a stent-valve includes an inner shaft having a distal end, a proximal end, and at least one lumen extending from the distal end to the proximal end, an expandable stent-valve configured to move between a compressed configuration and an expanded configuration, the stent-valve coupled to the inner shaft in the compressed configuration, the stent-valve having an upper portion including a plurality of arches and a plurality of radially outwardly extending upper anchoring crowns, a lower portion, and a valve, a distal sheath disposed over at least the lower portion of the stent-valve, a proximal sheath disposed over at least the upper portion of the stent-valve, and at least one cinching member coupled to the stent-valve and extending through one of the inner shaft, the distal sheath, or the proximal sheath, wherein the upper portion of the stent-valve is configured to move to the expanded configuration when the proximal sheath is withdrawn and the lower portion is configured to move to the expanded configuration when the distal sheath is withdrawn, wherein after at least a portion of the stent-valve has been moved to the expanded configuration, the at least one cinching member is configured to at least partially radially compress at least a portion of the stent-valve.

Alternatively or additionally to the embodiment above, the at least one cinching member is at least one suture coupled to the stent-valve.

Alternatively or additionally to any of the embodiments above, the suture has a middle region coupled to the stent-valve, and first and second free ends extending through at least one opening in the inner shaft and extending proximally through the inner shaft lumen and out a proximal end of the inner shaft, wherein pulling one or both free ends proximally causes the middle region to radially compress at least a portion of the heart-stent-valve.

Alternatively or additionally to any of the embodiments above, the middle region of the suture is positioned distal of the plurality of upper anchoring crowns.

Alternatively or additionally to any of the embodiments above, the lower portion of the stent-valve includes a plurality of struts forming a plurality of open cells, wherein the suture is coupled to at least two struts.

Alternatively or additionally to any of the embodiments above, the suture is a first suture positioned distal of the plurality of upper anchoring crowns, the delivery system further comprising a second suture coupled to the stent-valve, wherein the second suture is spaced apart from the first suture.

Alternatively or additionally to any of the embodiments above, wherein the at least one opening in the inner shaft includes a first opening positioned proximal of the distal end of the inner shaft, wherein at least one free end of the first suture extends through the first opening in the inner shaft, wherein at least one free end of the second suture extends through the distal end of the inner shaft, allowing the first and second sutures to be pulled independently or simultaneously to compress the lower portion of the stent-valve.

Alternatively or additionally to any of the embodiments above, the first and second sutures each extend around a circumference of the stent-valve.

Alternatively or additionally to any of the embodiments above, each of the first and second sutures extend around struts on opposite sides of the stent-valve.

Alternatively or additionally to any of the embodiments above, the suture includes first and second sutures each coupled to the stent-valve proximal of the upper crowns and extending proximally through the inner shaft lumen, the system further comprising third and fourth sutures coupled to at least two points of the distal end of the stent-valve and extending proximally through the inner shaft lumen.

Alternatively or additionally to any of the embodiments above, the delivery system further includes an outer shaft slidably disposed over the inner shaft, wherein the at last one suture includes first and second sutures each having a first end fixed to the inner shaft at a position proximal of the distal end of the inner shaft, and a second end coupled to the lower portion of the stent-valve, wherein when the outer shaft slides distally over the inner shaft, the outer shaft pulls the sutures radially inward causing the stent-valve to be compressed towards the inner shaft.

Alternatively or additionally to any of the embodiments above, the delivery system further includes a rod extending through the inner shaft, wherein the first and second free ends are twisted around the rod, such that rotating the rod pulls the middle region of the suture toward and into the inner shaft, thereby radially compressing the stent-valve.

Alternatively or additionally to any of the embodiments above, the inner shaft has an opening spaced proximally from the distal end of the inner shaft, wherein the first and second free ends of the suture are wrapped around an outer surface of the inner shaft proximally from the distal end and then extend into the opening, such that as the inner shaft is rotated, the suture wraps around the outer surface, shortening a middle region of the suture and pulling the stent-valve radially inward into the radially compressed configuration.

Alternatively or additionally to any of the embodiments above, the inner shaft has a distal tip, wherein the at least one suture includes a plurality of sutures each having a first end fixed to the distal tip, each suture coupled to the stent-valve and then extending into an opening in the inner shaft, the opening spaced proximally from the distal tip.

Alternatively or additionally to any of the embodiments above, the inner shaft has a distal tip, wherein the at least one cinching member includes a plurality of ribbons each having a first end fixed to the distal tip, the ribbon extending proximally and woven through the plurality of open cells and extending proximally along the inner shaft to the proximal end.

Alternatively or additionally to any of the embodiments above, the delivery system further includes an outer shaft slidably extending over the inner shaft, wherein the inner shaft has a distal tip, wherein the at least one cinching member includes a plurality of ribbons each having a first end fixed to the distal tip and a second end coupled to the outer shaft, the ribbon extending from the distal tip proximally and helically around an outer surface of the stent-valve, wherein the outer shaft is rotatable in an opposite direction as the inner shaft, thereby cinching the stent-valve as the ribbons contract radially inward.

Alternatively or additionally to any of the embodiments above, the inner shaft has a distal tip, wherein the lower portion of the stent-valve includes a plurality of struts forming a plurality of open cells, wherein at least some of the plurality of struts include distal extensions that converge into at least two paddles, wherein the at least one cinching member includes a capture member defined by the distal tip, the capture member configured to releasably engage the at least two paddles.

Alternatively or additionally to any of the embodiments above, the lower portion of the stent-valve includes a plurality of struts forming a plurality of open cells including lower open cells defining lower crowns, wherein the inner shaft has a distal tip, wherein the at least one cinching member includes a plurality of recapture members having first ends fixed to the distal tip and second ends defining hooks configured to releasably engage the lower open cells, wherein proximal movement of the distal sheath compresses the recapture members and the lower crowns radially inward, allowing the lower crowns to be re-inserted into the distal sheath.

Alternatively or additionally to any of the embodiments above, the lower portion of the stent-valve includes a plurality of struts forming a plurality of open cells including lower open cells defining a distal end of the stent-valve, wherein the at least one cinching member includes a plurality of hooks slidably extending from the distal end of the inner shaft, the hooks configured to engage the open cells and when pulled proximally, pull the distal end of the stent-valve into a radially contracted configuration.

Alternatively or additionally to any of the embodiments above, the lower portion of the stent-valve includes a plurality of struts forming a plurality of open cells including lower open cells defining lower crowns, wherein the at least one cinching member includes a plurality of release sheaths slidably extending out the distal end of the inner shaft, wherein a tether extends slidably through each of the plurality of release sheaths, out distal ends thereof and around at least one strut forming a bend in the tether, and a free distal end of the tether extends back into the release sheath, wherein each tether has a resting shape in which the bend is 90 degrees or less, wherein the tethers are configured such that when the release sheath is pulled proximally relative to the tether, once the free end of the tether is released from the release sheath, the tether returns to the resting shape thereby releasing the strut and allowing the tether to be withdrawn proximally from the stent-valve.

Alternatively or additionally to any of the embodiments above, the lower portion of the stent-valve includes a plurality of struts forming a plurality of open cells including lower open cells defining lower crowns, wherein the inner shaft has a distal tip, wherein the at least one cinching member includes an expandable mesh having a first end fixed to the distal tip and a second free end configured to extend over and surround the lower crowns, wherein the distal sheath is configured to be moved distally to allow the mesh and lower portion of the stent-valve to expand, and to be moved proximally to radially constrain the mesh and distal crowns.

Alternatively or additionally to any of the embodiments above, each of the plurality of upper anchoring crowns includes an extension connecting the upper crown to one of the plurality of arches, wherein the at least one cinching member includes a plurality of cinching members, each cinching member configured to extend distally from a delivery catheter and releasably engage one of the plurality of arches.

Alternatively or additionally to any of the embodiments above, the plurality of cinching members are sutures, wherein first and second free ends of each suture extend proximally through the delivery catheter and a middle portion of each suture forms a loop around one of the plurality of arches.

Alternatively or additionally to any of the embodiments above, the plurality of cinching members are hooks configured to extend distally from the distal end of the delivery catheter and releasably engage one of the plurality of arches.

Another delivery system configured to deliver, adjust, and/or recapture a stent-valve includes an inner shaft having a distal end, a proximal end, and at least one lumen extending from the distal end to the proximal end, an expandable stent-valve configured to move between a compressed configuration and an expanded configuration, the stent-valve coupled to the inner shaft in the compressed configuration, the stent-valve having an upper portion including a plurality of arches and a plurality of radially outwardly extending upper anchoring crowns, a lower portion, and a valve, the lower portion including a plurality of struts defining a plurality of open cells, a distal sheath disposed over at least the lower portion of the stent-valve a proximal sheath disposed over at least the upper portion of the stent-valve, and at least one cinching member coupled to the stent-valve and extending through one of the inner shaft, the distal sheath, or the proximal sheath, wherein the at least one cinching member is configured to gradually radially compress and release at least a portion of the stent-valve in the absence of the proximal and distal sheaths.

An example method of delivering and repositioning a stent-valve includes the step of inserting a distal tip of a stent-valve delivery system through a patient's aorta and aortic valve, the delivery system including an inner shaft including the distal tip, a stent-valve crimped onto the inner shaft, the stent-valve including an upper portion, a lower portion, and a valve, a distal sheath disposed over at least the lower portion of the stent-valve, a proximal sheath disposed over at least the upper portion of the stent-valve, and at least one cinching member coupled to at least the lower portion of the stent-valve and extending through one of the inner shafts, the distal sheath, or the proximal sheath. The method further includes the steps of moving the proximal sheath proximally to release the upper portion of the stent-valve, moving the distal sheath distally to release the lower portion of the stent-valve, cinching the cinching member thereby radially compressing at least the lower portion of the stent-valve, repositioning at least the lower portion of the stent-valve, and releasing the cinching member thereby allowing the lower portion of the stent-valve to expand.

The above summary of some embodiments, aspects, and/or examples is not intended to describe each embodiment or every implementation of the present disclosure. The figures and the detailed description which follows more particularly exemplify these embodiments.

### BRIEF DESCRIPTION OF THE DRAWINGS

The disclosure may be more completely understood in consideration of the following detailed description of various embodiments in connection with the accompanying drawings, in which:

FIG. 1 illustrates a prior art replacement stent-valve;

FIG. 2 illustrates a delivery system disposed within the aortic arch and aortic valve;

FIG. 3 illustrates the delivery system of FIG. 2 with the proximal sheath withdrawn from the stent-valve;

FIG. 4 illustrates the delivery system of FIG. 2 with the distal sheath withdrawn from the stent-valve and the stent-valve fully expanded;

FIG. 5 is a partial cross-sectional view of the stent portion of a stent-valve with an example cinching member;

FIG. 6A is a partial cross-sectional view of a stent-valve and another cinching member;

FIG. 6B is a bottom view of the stent-valve and cinching member of FIG. 6A;

FIGS. 7A-17 are partial cross-sectional views of a stent-valve and additional example cinching members;

FIGS. 18A and 18B are partial cross-sectional views of a prior art stent from a stent-valve in an expanded configuration and partially withdrawn into a catheter, respectively;

FIGS. 19A and 19B are partial cross-sectional views of example modified stent-valves and cinching members;

FIGS. 20A-20C are side views showing a modified stent-valve being pulled into a catheter;

FIG. 21 is a partial cross-sectional view of a stent-valve and claw device;

FIG. 22 is a side view of the claw device of FIG. 21;

FIGS. 23A and 23B are side cross-sectional and top views, respectively, of an example wire holder;

FIGS. 24A-24F are side views of a portion of a wire and hook;

FIGS. 25-27A are partial cross-sectional views of stent-valves with example centering members;

FIG. 27B is a top view of the stent-valve and centering member of FIG. 27A;

FIG. 28A is a partial cross-sectional view of another modified stent-valve;

FIG. 28B illustrates a portion of the upper crown of the modified stent-valve of FIG. 28A in a withdrawal configuration; and

FIG. 29 illustrates another example stent-valve.

While aspects of the disclosure are amenable to various modifications and alternative forms, specifics thereof have been shown by way of example in the drawings and will be described in detail. It should be understood, however, that the intention is not to limit aspects of the disclosure to the particular embodiments described. On the contrary, the intention is to cover all modifications, equivalents, and alternatives falling within the spirit and scope of the disclosure.

### DETAILED DESCRIPTION

For the following defined terms, these definitions shall be applied, unless a different definition is given in the claims or elsewhere in this specification.

All numeric values are herein assumed to be modified by the term “about,” whether or not explicitly indicated. The term “about”, in the context of numeric values, generally refers to a range of numbers that one of skill in the art would consider equivalent to the recited value (e.g., having the same function or result). In many instances, the term “about” may include numbers that are rounded to the nearest significant figure. Other uses of the term “about” (e.g., in a context other than numeric values) may be assumed to have their ordinary and customary definition(s), as understood from and consistent with the context of the specification, unless otherwise specified.

The recitation of numerical ranges by endpoints includes all numbers within that range, including the endpoints (e.g., 1 to 5 includes 1, 1.5, 2, 2.75, 3, 3.80, 4, and 5). Although some suitable dimensions, ranges, and/or values pertaining to various components, features and/or specifications are disclosed, one of skill in the art, incited by the present disclosure, would understand desired dimensions, ranges, and/or values may deviate from those expressly disclosed.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise. It is to be noted that in order to facilitate understanding, certain features of the disclosure may be described in the singular, even though those features may be plural or recurring within the disclosed embodiment(s). Each instance of the features may include and/or be encompassed by the singular disclosure(s), unless expressly stated to the contrary. For simplicity and clarity purposes, not all elements of the disclosure are necessarily shown in each figure or discussed in detail below. However, it will be understood that the following discussion may apply equally to any and/or all of the components for which there are more than one, unless explicitly stated to the contrary. Additionally, not all instances of some elements or features may be shown in each figure for clarity.

Relative terms such as “proximal”, “distal”, “advance”, “withdraw”, variants thereof, and the like, may be generally considered with respect to the positioning, direction, and/or operation of various elements relative to a user/operator/manipulator of the device, wherein “proximal” and “withdraw” indicate or refer to closer to or toward the user and “distal” and “advance” indicate or refer to farther from or away from the user. In some instances, the terms “proximal” and “distal” may be arbitrarily assigned in an effort to facilitate understanding of the disclosure, and such instances will be readily apparent to the skilled artisan. Other relative terms, such as “upstream”, “downstream”, “inflow”, and “outflow” refer to a direction of fluid flow within a lumen, such as a body lumen, a blood vessel, or within a device.

The term “extent” may be understood to mean a greatest measurement of a stated or identified dimension, unless the extent or dimension in question is preceded by or identified as a “minimum”, which may be understood to mean a smallest measurement of the stated or identified dimension. For example, “outer extent” may be understood to mean a maximum outer dimension, “radial extent” may be understood to mean a maximum radial dimension, “longitudinal extent” may be understood to mean a maximum longitudinal dimension, etc. Each instance of an “extent” may be different (e.g., axial, longitudinal, lateral, radial, circumferential, etc.) and will be apparent to the skilled person from the context of the individual usage. Generally, an “extent” may be considered a greatest possible dimension measured according to the intended usage, while a “minimum extent” may be considered a smallest possible dimension measured according to the intended usage. In some instances, an “extent” may generally be measured orthogonally within a plane and/or cross-section, but may be, as will be apparent from the particular context, measured differently-such as, but not limited to, angularly, radially, circumferentially (e.g., along an arc), etc.

The terms “monolithic” and “unitary” shall generally refer to an element or elements made from or consisting of a single structure or base unit/element. A monolithic and/or unitary element shall exclude structure and/or features made by assembling or otherwise joining multiple discrete elements together.

It is noted that references in the specification to “an embodiment”, “some embodiments”, “other embodiments”, etc., indicate that the embodiment(s) described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it would be within the knowledge of one skilled in the art to affect the particular feature, structure, or characteristic in connection with other embodiments, whether or not explicitly described, unless clearly stated to the contrary. That is, the various individual elements described below, even if not explicitly shown in a particular combination, are nevertheless contemplated as being combinable or arrangeable with each other to form other additional embodiments or to complement and/or enrich the described embodiment(s), as would be understood by one of ordinary skill in the art.

For the purpose of clarity, certain identifying numerical nomenclature (e.g., first, second, third, fourth, etc.) may be used throughout the description and/or claims to name and/or differentiate between various described and/or claimed features. It is to be understood that the numerical nomenclature is not intended to be limiting and is exemplary only. In some embodiments, alterations of and deviations from previously-used numerical nomenclature may be made in the interest of brevity and clarity. That is, a feature identified as a “first” element may later be referred to as a “second” element, a “third” element, etc. or may be omitted entirely, and/or a different feature may be referred to as the “first” element. The meaning and/or designation in each instance will be apparent to the skilled practitioner.

The following description should be read with reference to the drawings, which are not necessarily to scale, wherein similar elements in different drawings are numbered the same. The detailed description and drawings are intended to illustrate but not limit the disclosure. Those skilled in the art will recognize that the various elements described and/or shown may be arranged in various combinations and configurations without departing from the scope of the disclosure. The detailed description and drawings illustrate example embodiments of the disclosure. However, in the interest of clarity and ease of understanding, while every feature and/or element may not be shown in each drawing, the feature(s) and/or element(s) may be understood to be present regardless, unless otherwise specified.

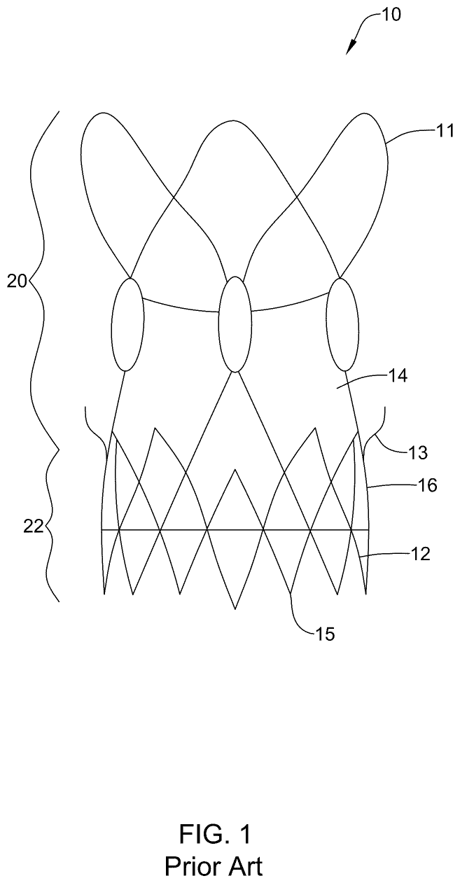

FIG. 1 illustrates a prior art aortic replacement stent-valve 10 including valve leaflets 14 attached to an expandable stent 12. The stent 12 includes an upper stent portion 20 with a plurality of support arches 11 defining a proximal or downstream end of the stent-valve 10, and a plurality of radially outwardly extending upper anchoring crowns 13. The stent 12 includes and a lower stent portion 22 including the stent portion extending distal of the upper anchoring crowns 13 and including a plurality of lower crowns 15 defining a distal or upstream end of the stent-valve 10. The upper anchoring crowns 13 define outwardly protruding stent regions 13 designed to engage the native valve leaflets to prevent migration of the prosthetic stent-valve 10. The outwardly protruding stent regions 13 may be formed by bending and heat setting upper portions of some stent regions of the stent structure 12. In some embodiments, a waist region 16 may be defined distal of the upper anchoring crowns 13, having a smaller outer diameter than the widest part of the lower stent portion 22. The arches 11, upper anchoring crowns 13, and lower crowns 15 act as anchoring structures within the native aortic annulus for the valve 14. The entire stent structure 12 may be self-expandable with the arches 11 and lower stent portion 22 being separately self-expandable during delivery when removed separately from constraining sheaths.

When implanting a TAVR type valve, proper positioning is critical for the proper functioning of the valve. Self-expanding TAVR valves often contain nitinol frames that are elastically compressed onto the delivery system and are released to expand into the aortic valve during the procedure. The proper position of the valve must be determined while valve is mostly compressed on delivery system. In most TAVR systems, the valve release from the delivery system occurs in less than a second. After release, readjustment, repositioning, recapture, or removal of the valve is often impossible without surgery. Improper positioning during delivery may result in ventricular or aortic migration which could cause serious complications for the patient. In many cases, the delivery system for implanting a TAVR type valve lacks features that allow physicians to adjust, reposition, recapture, or bail out with the implant after it is released from the delivery system.

Adding features to the delivery system and/or the implant that would give physicians full control of implant diameter during deployment increases the likelihood of properly positioning the valve during delivery. A controlled deployment may also allow for reversible expansion, and thus allow for repositioning if initial placement is not ideal. This may occur after only the upper portion 20 has been released or after complete deployment of the stent-valve 10, including both the upper portion 20 and the lower portion 22.

Further, providing features to the delivery system that allow the valve to be pulled out proximally without causing damage to the patient's anatomy is advantageous when it is desired to reverse the entire delivery process, particularly when the removal and bail out of the procedure can occur rapidly. The delivery systems described below provide various structures that allow for controlled expansion, adjustment, repositioning, and removal of the stent-valve after at least partial deployment of the stent-valve.

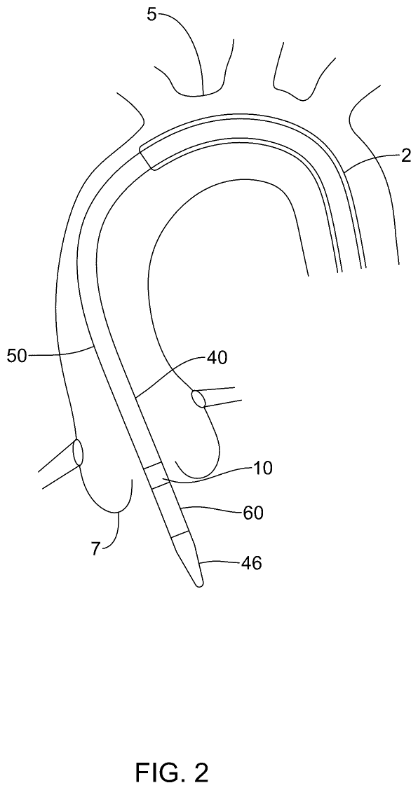

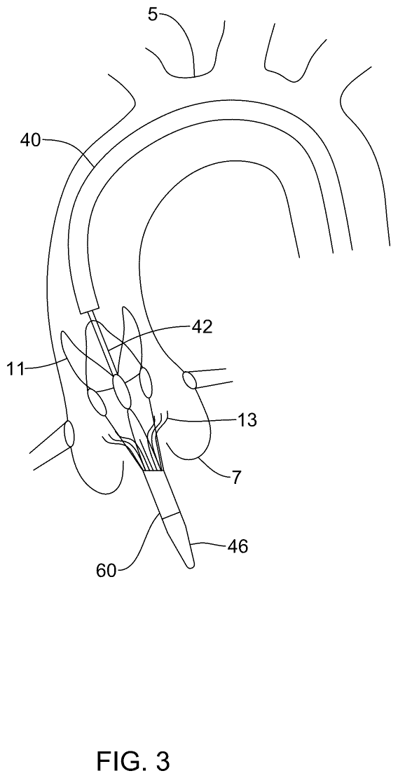

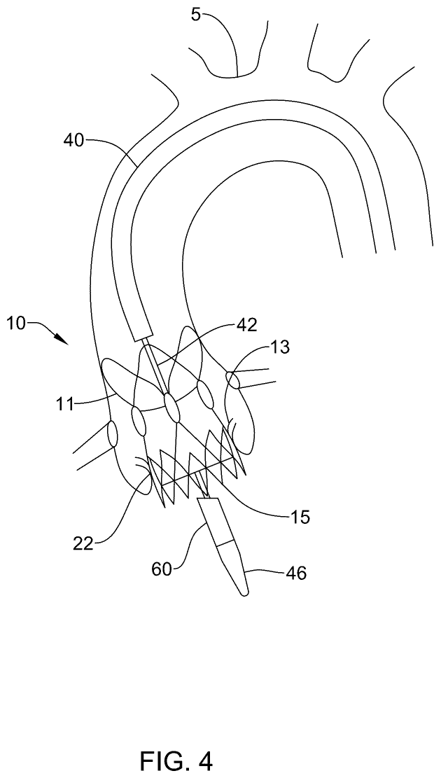

FIG. 2 illustrates a conventional stent-valve delivery system during a transfemoral access method. The delivery system 50 is inserted through a catheter 2 through the femoral artery and vasculature, across the aortic arch 5 and through the aortic valve 7. The delivery system 50 may include a proximal sheath 40 and a distal sheath 60 constraining the stent-valve 10. The stent-valve 10 may be configured to move between a compressed configuration and an expanded configuration. The stent-valve may be coupled to the inner shaft in the compressed configuration, proximal of a distal tip 46 fixed to the inner shaft 42. In a first step during deployment, the proximal sheath 40 may be withdrawn proximally to release the upper portion 20 of the stent structure 12 including the plurality of arches 11 and upper anchoring crowns 13, as shown in FIG. 3. The lower crowns 15 remain constrained within the distal sheath 60. A second step during deployment is shown in FIG. 4, in which the distal sheath 60 is moved distally off the lower stent portion 22 including the lower crowns 15, allowing the stent-valve to fully expand against the aortic valve 7 with the arches 11 expanding against the inner wall of the aorta. The delivery system including the proximal sheath 40, distal sheath 60, inner shaft 42, and distal tip 46 is then withdrawn through the implanted stent-valve 10 and removed from the body.

The conventional delivery system and method results in rapid expansion and seating of the stent-valve 10, without opportunity for modification of the position of the stent-valve. For example, the proximal sheath 40 may be withdrawn from the upper anchoring crowns 13 and the arches 11 in one movement, resulting in the arches 11 and upper anchoring crowns 13 being expanded radially outward beyond the inner diameter of the proximal sheath 40, as shown in FIG. 3. Once the distal sheath 60 is moved distally off the lower crowns 15, the lower portion 22 of the stent structure 12 may pop open resulting in the entire stent-valve 10 expanding into the fully deployed configuration as shown in FIG. 4. As a result, there may not be any way to control the deployment, adjust the position of the stent-valve 10 after it has been partially deployed, recapture one or more portions of the stent-valve 10, or bail out of the procedure and completely remove the stent-valve 10.

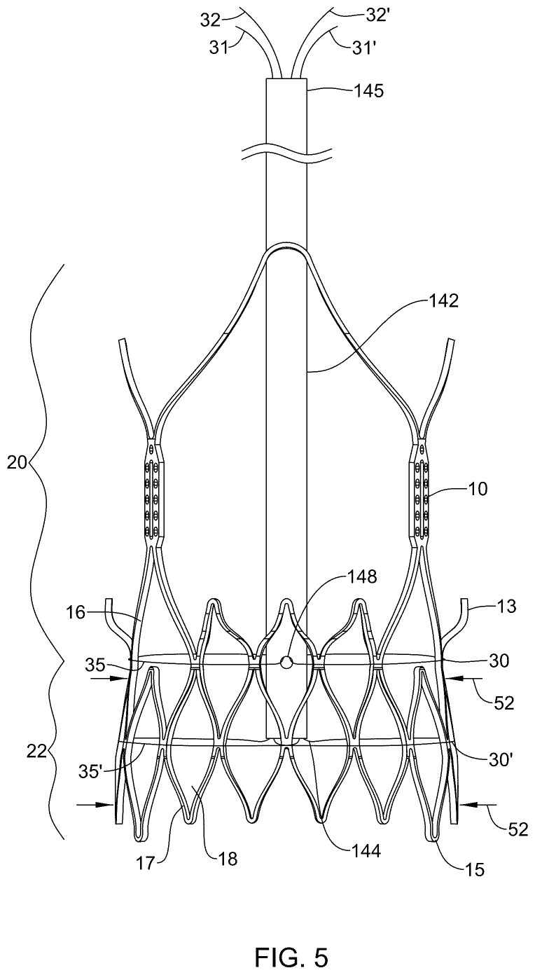

A number of different delivery systems and structures are described below, which allow for a controlled delivery of the stent-valve 10, allowing for radially compressing at least a portion of the stent-valve after the stent-valve has been fully expanded. This will allow for the stent-valve to be repositioned after deployment. Additionally, the delivery systems may provide for a slower and more controlled expansion of the stent-valve 10 compared to the near instantaneous prior art delivery systems and methods. In the delivery systems described below and shown in FIGS. 5-28, the stent-valve 10, proximal sheath 40, and distal sheath 60 are as described above, unless otherwise noted and described. The valve of the stent-valve has been removed from the figures to better illustrate the elements of the delivery system. In each embodiment shown in FIGS. 5-17, one or more cinching or compressing members allow for the gradual expansion of one or more parts of the stent-valve once the proximal and/or distal sheaths 40, 60 have been withdrawn. The cinching or compressing members also allow for the upper and/or lower portions 20, 22 of the stent-valve 10 to be at least partially compressed to allow for readjustment or repositioning of the stent-valve after expansion.

FIG. 5 illustrates a delivery system configured to deliver, adjust, and/or recapture the stent-valve 10. The valve portion of the stent-valve has been removed to better illustrate the elements of the delivery system. As shown, the inner shaft 142 has a distal end 144, proximal end 145, and an opening 148 extending through a side wall into the lumen that extends from the distal end 144 to the proximal end 145. At least one cinching member 30 may be coupled to the stent-valve 10 and extend through one of the inner shafts 142, the distal sheath 60, or the proximal sheath 40. The cinching member 30 is configured such that after the proximal sheath 40 is withdrawn proximally to release the upper portion 20 of the stent structure 12 including the plurality of arches 11 and upper anchoring crowns 13, as shown in FIG. 3, the cinching member 30 may be actuated to control and gradually allow for the expansion of the lower portion 22 of the stent-valve 10 during and after the distal sheath 60 is moved distally to uncover the lower portion of the stent-valve. Additionally, after the stent-valve 10 has fully expanded, the cinching member 30 may be actuated to at least partially radially compress at least a portion of the stent-valve, as indicated by arrows 52, to allow for the position of the stent-valve 10 to be readjusted.

In the embodiment shown in FIG. 5, the cinching members 30 are sutures coupled to the stent-valve. Each suture 30 may have a middle region 35 coupled to the stent-valve, and a first free end 31 and a second free end 32 extending through an opening in the inner shaft and proximally through the lumen in the inner shaft 142, and extending out the proximal end 145 of the inner shaft 142. The suture 30 may be actuated by pulling one or both of the first and second free ends 31, 32 proximally which causes the middle region 35 to radially compress at least a portion of the stent-valve 10. The free ends 31, 32 may be coupled to a pulling member, such as a spool around which the suture may be wound to compress the middle region 35, to a rod slidable within the proximal region of the inner shaft, or any other structure configured to pull the free ends of the suture proximally and hold tension on the suture to radially compress the stent-valve while it is repositioned or adjusted. For example, the sutures could be attached to the delivery system handle, which either wraps up or translates the sutures with a lead screw or other mechanism. The sutures would be accessible either directly on the handle or extending from the handle in order to cut or otherwise remove the sutures upon completion of the delivery method.

Alternatively, the free ends of the suture may be manually pulled to compress the stent-valve, and then the free ends may be secured or locked against further axial movement, such as with a spring clip at the proximal end of the delivery catheter. To control the expansion of the lower portion 22 of the stent-valve during deployment, the free ends 31, 32 of the suture 30 may be pulled and held under tension as the distal sheath is removed from the stent-valve. This prevents the lower portion of the stent-valve from expanding rapidly as soon as the distal sheath 60 moves off the lower crowns 15. The suture 30 may be gradually released to allow for a gradual expansion of the lower portion 22 of the stent-valve, with readjustment of its position, if necessary. After adjusting or repositioning the stent-valve, when the stent-valve is fully expanded, one end of the suture may be released to pull the entire suture proximally out of the delivery system or if the ends of the suture are locked in place, the suture may be cut to allow for removal.

The degree of compression of the stent-valve 10 may be adjusted by pulling more or less of the suture free ends 31, 32 proximally out of the inner shaft 142. This may allow a fully expanded stent-valve 10 to be repositioned or readjusted. Once the stent-valve has been sufficiently radially compressed to release it from the native valve, the stent-valve may be repositioned. Once the stent-valve is in the desired position, the first free end 31 of the suture 30 may be released and the second 32 free end may be pulled proximally such that the free end 31 is pulled distally through the inner shaft 142 and then around the stent-valve 10 and back proximally through the inner shaft 142 to be completely removed from the inner shaft 142. In some embodiments, the first and second free ends 31, 32 of the suture 30 may be pulled tight enough such that the lower portion 22 of the stent-valve 10 is compressed until the distal sheath 60 can move proximally over at least the lower crowns 15. The stent-valve may then be moved into a more desirable position, at which time, the distal sheath may be moved distally and the suture removed as described above.

In the embodiment shown in FIG. 5, the middle region 35 of a first suture 30 is coupled to the lower portion of the stent-valve, distal of the plurality of upper anchoring crowns 13. When the stent-valve 10 has a waist region 16, the suture 30 may be disposed in the waist region 16. In some embodiments, the suture 30 may extend at least partially around the circumference of the stent-valve. In other embodiments, the suture 30 may extend completely around the circumference of the stent-valve. As illustrated in FIG. 5, the delivery system may include a second suture 30′ coupled to the stent-valve, spaced apart axially from the first suture 30. As shown, the middle region 35′ of the second suture 30′ is spaced apart distally from the first suture 30. The first and second free ends 31′, 32′ of the second suture 30′ may extend out of the proximal end 145 of the inner shaft 142. When two or more sutures are present, all of the sutures may be actuatable simultaneously, each suture may be separately and independently actuatable, or groups of sutures may be actuatable together while other sutures remain unactuated. The free ends of all sutures may extend into the inner shaft lumen through the opening at the distal end 144. In other embodiments, the first and second free ends 31, 32 of a first suture 30 may extend through an opening 148 extending through a side wall of the inner shaft 142, where the opening 148 is spaced proximally from the distal end 144, and the first and second free ends 31′, 32′ of a second suture 30′ extend through the distal end 144 of the inner shaft 142, as shown in FIG. 5. This allows the first and second sutures 30, 30′ to be pulled independently or simultaneously to compress the lower portion of the stent-valve 10.

The lower portion of the stent-valve 10 may include a plurality of struts 17 forming a plurality of open cells 18, and the suture 30 may be coupled to at least two struts. As illustrated in FIG. 5, suture 30 may be interwoven into and out of the plurality of open cells 18 such that the middle region 35 extends at least part way around the circumference of the stent-valve 10.

In another embodiment, the first end of the suture may be fixed to the stent-valve 10, and the remainder of the suture may extend around at least a portion of the circumference of the stent-valve with the free second end extending through the lumen of the inner shaft 142 and out the proximal end 145. The free end of the suture may be pulled proximally to cinch the stent-valve, and once the stent-valve has been repositioned or readjusted, the first end of the suture may be cut with a cutting implement extending through the inner shaft 142 or through the delivery catheter.

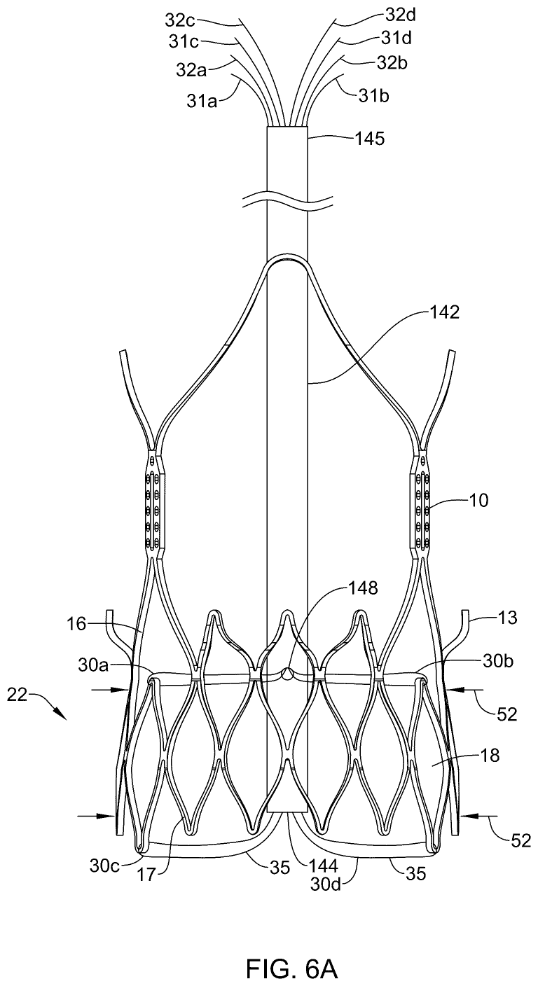

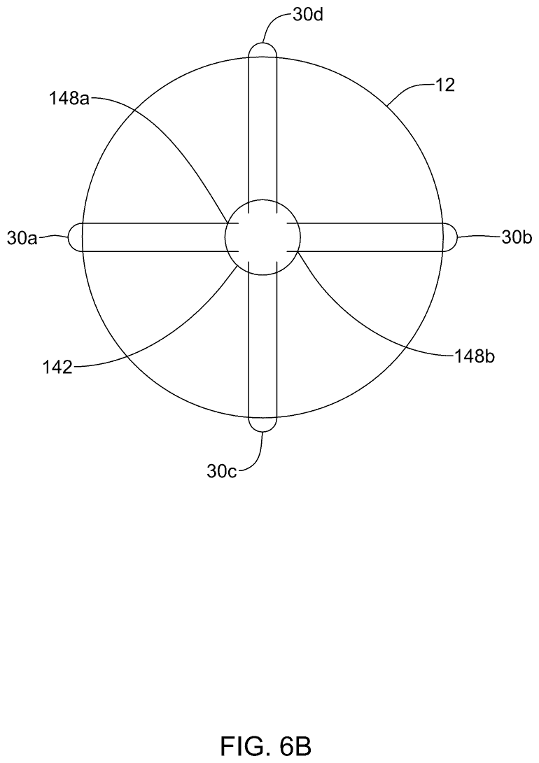

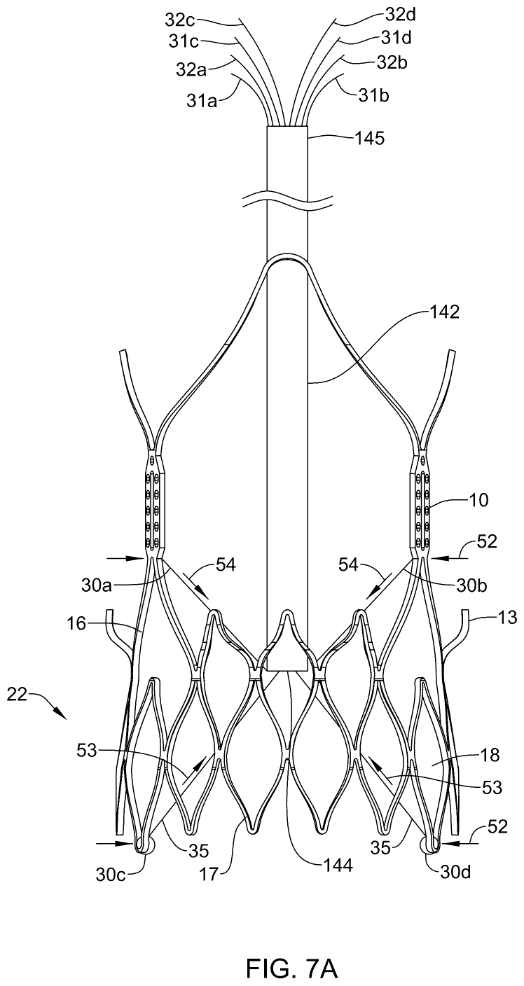

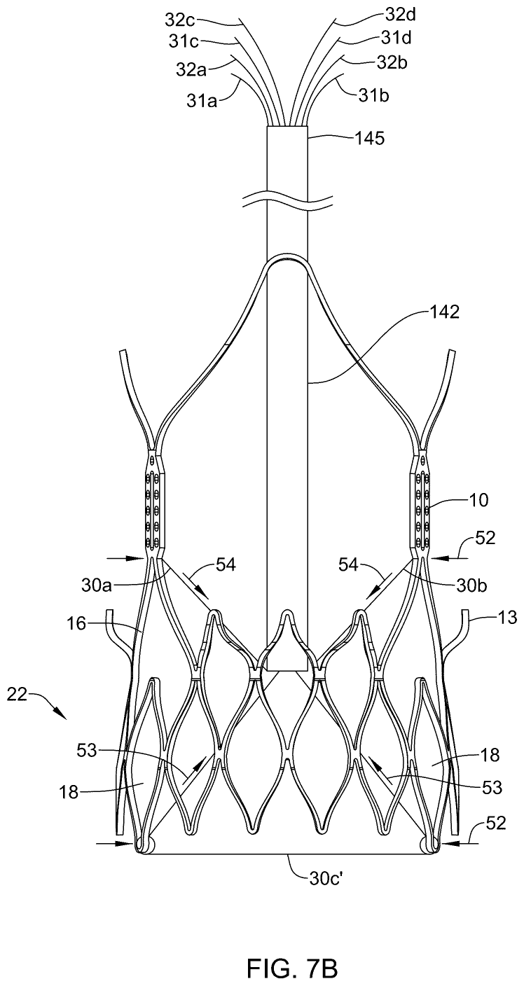

FIGS. 6A, 6B, and 7A illustrate embodiments in which the cinching member includes first, second, third, and fourth sutures 30a, 30b, 30c, 30d (collectively sutures 30) which each have a middle region 35 coupled to the stent-valve 10, with free first ends 31a, 31b, 31c, 31d (collectively 31) and free second ends 32a, 32b, 32c, 32d (collectively 32) extending proximally through the inner shaft 142 and exiting the proximal end 145 of the inner shaft. In the embodiment shown in FIGS. 6A and 6B, the sutures 30 are coupled to one or two struts 17 of the lower portion 22 of the stent-valve 10. The first and second sutures 30a, 30b are coupled to struts 17 on opposing sides of the stent-valve, and the third and fourth sutures 30c, 30d are coupled to struts 17 on opposing sides of the stent-valve. In the embodiment shown in FIG. 6A, the first and third sutures 30a, 30c are coupled to a first side of the stent 12 and the second and fourth sutures 30b, 30d are coupled to a second side of the stent 12 opposite the first side. FIG. 6B shows a view looking down the length of the stent 12 from the distal end, with the upper anchoring crowns 13 and valve 14 not shown. The first and second sutures 30a, 30b lie in a first plane and the third and fourth sutures 30c, 30d lie in a second plane perpendicular to the first plane. In this manner, the four sutures 30, when pulled proximally, provide a compressive force at four points around the circumference of the stent-valve. In other embodiments, three sutures may be attached to the stent-frame at positions roughly a third of the way around the circumference from one another (not shown). Additionally, more than four sutures may be coupled to spaced-apart locations around the circumference of the stent-valve. The sutures 30 may be attached to the stent-valve 10 at positions such that pulling on the sutures simultaneously or sequentially, will result in the radial compression of the stent-frame, as indicated by arrows 52.

The free ends 31, 32 of all of the sutures 30 may enter the lumen of the inner shaft 142 through the distal end 144. Alternatively, some of the sutures 30 may extend into the distal end 144 while other sutures may enter one or more opening 148 through the side wall of the inner shaft 142 proximal of the distal end 144, as shown in FIG. 6A. In some embodiments, two openings 148a, 148b may be positioned on opposite sides of the inner shaft 142, with one suture extending through each opening 148a, 148b, as shown in FIG. 6B.

In a further embodiment, the first and second sutures 30a, 30b may be coupled to a portion of the stent-valve proximal of the upper anchoring crowns 13, and the third and fourth sutures 30c, 30d, may be coupled to struts 17 at the distal end of the stent-valve 10 as shown in FIG. 7A. In this embodiment, all four sutures 30 may enter the inner shaft lumen through the distal end 144. The third and fourth sutures 30c, 30d coupled to the distal end may apply a radially inward and proximal force on the stent-valve, indicated by arrows 53 while the first and second sutures 30a, 30b coupled proximal of the upper anchoring crowns 13 may apply a radially inward and distal force on the stent-valve, indicated by arrows 54, which together provides a compressive force on the stent-valve, causing the stent-valve 10 to radially compress. Similar to FIG. 6B, the first and second sutures 30a, 30b in the embodiment of FIG. 7A may lie in a first plane and the third and fourth sutures 30c, 30d may lie in a second plane perpendicular to the first plane.

In the embodiments shown in FIGS. 6A, 6B, and 7A, the middle region 35 of each suture is shown extending through a single open cell 18 formed by adjacent struts 17. However, in an alternative configuration, the middle region 35 of each suture may extend through two or more adjacent open cells 18, but not around the complete circumference of the stent-valve 10. As shown in FIG. 7B, which is similar to FIG. 7A except that the middle region 35 of the third suture 30c′ extends through two open cells 18 and then into the inner shaft 142. As illustrated, the open cells 18 are spaced apart, giving the middle region 35 a triangular shape and allowing for opposing sides of the stent-valve 10 to be pulled towards the inner shaft as the stent-valve is compressed. In other embodiments, the open cells 18 through which the suture extends may be adjacent.

FIG. 8 illustrates a further embodiment in which the cinching member includes a first suture 130a and a second suture 130b, and in which an outer shaft 141 may be slidably disposed over the inner shaft 142 to compress the sutures and provide a radially inward compressing force on the stent-valve. First and second sutures 130a, 130b may each have a first end 133a, 133b fixed to the inner shaft 142 at a position proximal of the distal end 144 of the inner shaft, and a second end 134a, 134b coupled to one or more struts 17 in the lower portion of the stent-valve 10. When the outer shaft 141 slides distally over the inner shaft 142, the outer shaft pulls the sutures 130a, 130b radially inward causing the stent-valve to be compressed towards the inner shaft, as indicated by arrows 56. The second ends 134a, 134b of the sutures may be coupled to opposing sides of the stent-valve 10, as shown in FIG. 8, to provide a radially inward force on opposing sides of the stent-valve to achieve substantially even compression of the stent-valve, as indicated by arrows 52.

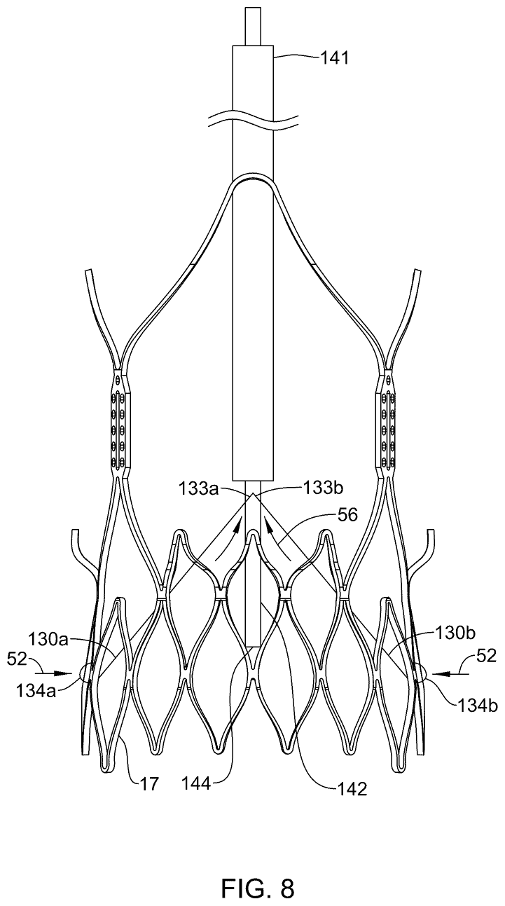

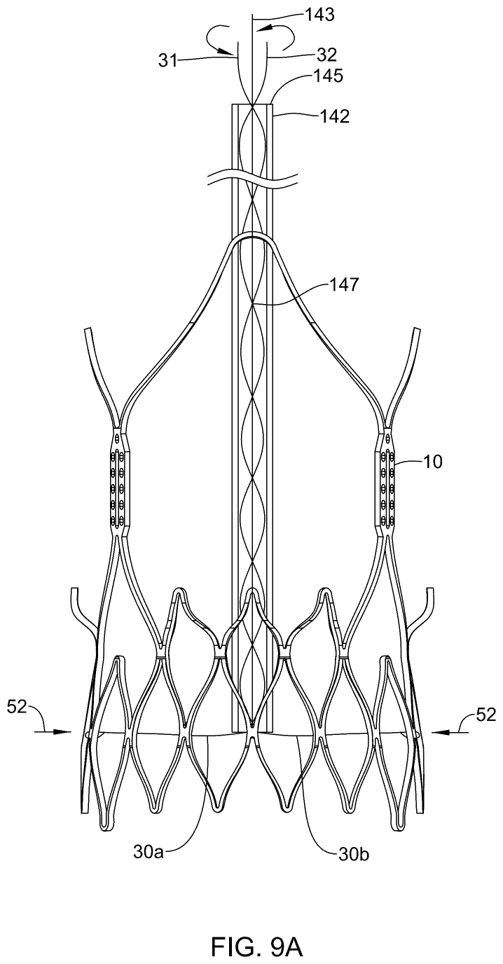

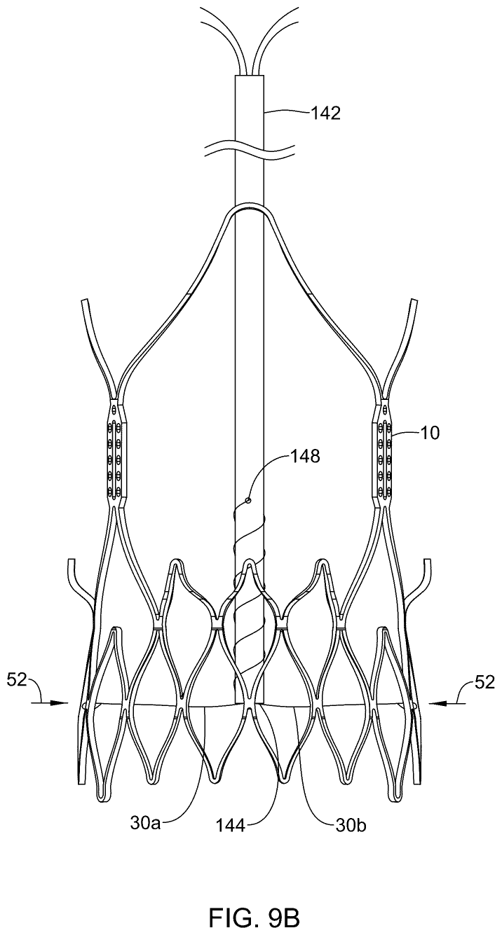

FIGS. 9A and 9B illustrate embodiments in which the cinching member includes at least one suture 30 coupled to the stent-valve 10 that may be retracted rotationally. The embodiment in FIG. 9A is similar to that shown in FIGS. 5 and 6A, except that a rod 143 is disposed through the lumen 147 of the inner shaft 142 and the free ends 31, 32 of the suture 30 are wrapped around the rod 143 as they extend proximally through the inner shaft 142 and exit the proximal end 145. The at least one suture 30 may be a single suture 30 extending around the circumference of the stent-valve 10, as described above with regard to FIG. 5, or the at least one suture may include first and second sutures 30a, 30b coupled to opposing sides of the stent-valve, as described with regard to FIG. 6A. In both embodiments, the free ends 31, 32 of the suture(s) 30 are wrapped helically around the rod 143 and fixed to the rod. As the rod 143 is rotated, the suture is wound around the outside of the rod, changing the effective length of the suture and pulling the middle region 35 radially inward toward and into the inner shaft 142, resulting in radial compression of the stent-valve 10, as indicated by arrows 52.

FIG. 9B illustrates an alternative embodiment in which the inner shaft 142 has an opening 148 spaced proximally from the distal end 144 of the inner shaft, and the first and second free ends 31, 32 of the suture 30 are wrapped around the outer surface of the inner shaft proximally from the distal end and then extend into the opening 148. As the inner shaft 142 is rotated, the suture 30 wraps around the outer surface, shortening the middle region 35 and pulling the stent-valve radially inward into the radially compressed configuration, as indicated by arrows 52.

FIG. 10 illustrates another embodiment in which the cinching member includes a plurality of cinching members 230 each having a first end fixed to a distal tip 146 of the inner shaft 142. The second free end 232 of each cinching member 230 is coupled to the stent-valve and then extending into an opening 148 in the sidewall of the inner shaft 142, where the opening 148 is spaced proximally from the distal tip 146. In some embodiments, the cinching members 230 are coupled to the stent-valve by extending through an open cell 18 defined by the struts 17, as shown in FIG. 10. When the free ends 232 of the cinching members 230 are pulled proximally, the sutures apply a radially inward and proximal force on the stent-valve, thereby radially compressing the stent-valve, as indicated by arrows 52. The cinching members 230 may be sutures, wires, or cables.

In each of the embodiments shown in FIGS. 5-10 and described above, additional cinching members 30, 130, 230 may be wrapped around the arches 11, providing a cinching or compression force on the upper portion 20 of the stent-valve. Such additional sutures may have the same actuation mechanism as described above with regard to compressing the lower portion 22 of the stent-valve.

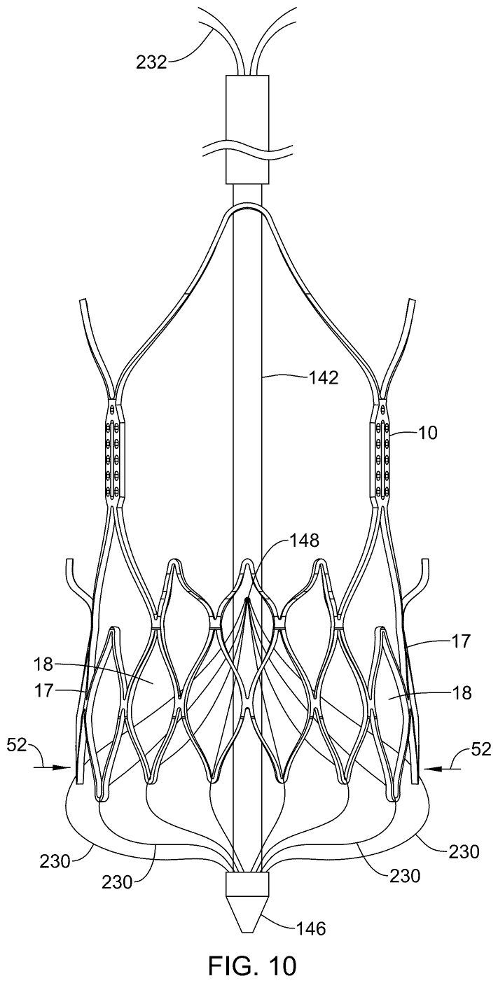

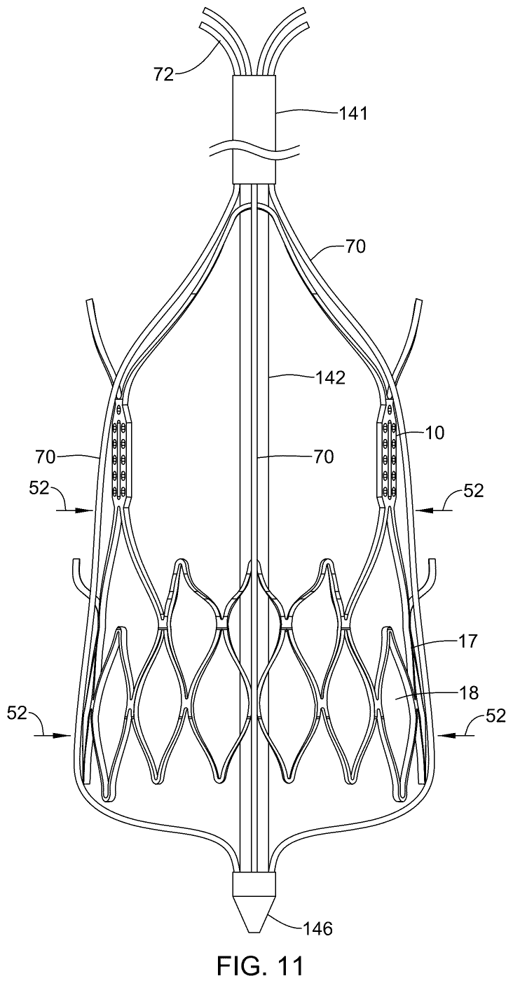

In a further embodiment, the cinching member may include a plurality of ribbons 70 configured to apply a radially inward compressing force on the stent-valve, as shown in FIGS. 11 and 12. Each of the ribbons 70 may have a first end fixed to the distal tip 146 of the inner shaft 142, with the second free end 72 extending proximally along the entire length of the stent-valve 10 and into an outer shaft 141 configured to slidably extend over the inner shaft 142, as shown in FIG. 11. The free ends 72 of the ribbons 70 may extend out the proximal end of the outer shaft 141. The ribbons 70 may be woven into and out of the open cells 18 formed by struts 17 of the stent-valve 10. Alternatively, the ribbons 70 may extend along the outer surface of the stent-valve 10 without being woven into the open cells. Pulling the free ends 72 of the ribbons proximally relative to the inner shaft 142 applies a radially inward compressive force on the entire stent-valve, thereby radially compressing both the upper and lower portions of the stent-valve 10, as indicated by arrows 52. In this way, holding tension on the ribbons 70 may allow for the entire stent-valve 10 to be gradually expanded after the proximal and distal shafts have been withdrawn. Additionally, pulling the free ends 72 of the ribbons proximally relative to the inner shaft 142 after the stent-valve 10 has fully expanded, may at least partially compress the entire stent-valve, allowing for readjustment or repositioning. After the stent-valve 10 has been compressed and moved into a more desirable position, the free ends 72 of the ribbons 70 may be released and the delivery system may be pulled out of the stent-valve. Alternatively, the ribbons may be cut adjacent where they are fixed to the distal tip 146.

The embodiment shown in FIG. 12 is similar to that shown in FIG. 11 except the cinching member includes a plurality of ribbons 70 extending from the distal tip 146 proximally and helically around the stent-valve, and the second ends of the ribbons may be fixed to the outer shaft 141 such that when the outer shaft 141 and inner shaft 142 are rotated in opposite directions, as indicated by arrows 57, the ribbons 70 are tightened helically around the stent-valve, thereby applying a radially inward compressive force on the stent-valve to radially compress the stent-valve 10, as indicated by arrows 52. The ribbons 70 may be wrapped around the outer surface of the stent-valve 10 or one or more of the ribbons 70 may be interwoven through at least one open cell 18. At least two ribbons 70 may be used, and in some embodiments, three, four, five, or more ribbons may be used. After the stent-valve 10 has been compressed and moved into a more desirable position, the ribbons 70 may be cut adjacent where they are fixed to the distal tip 146 and the delivery system may be pulled out of the stent-valve.

In addition to the cinch members for controlling delivery of the stent-valve discussed above, the delivery system may include cinching or compressing members that allow the distal end of the stent-valve, including the lower crowns 15, to be recaptured into the distal sheath 60 for axial adjustment of the stent-valve 10. The controlled delivery structures described above may be used in combination with the recapturing structures described below.

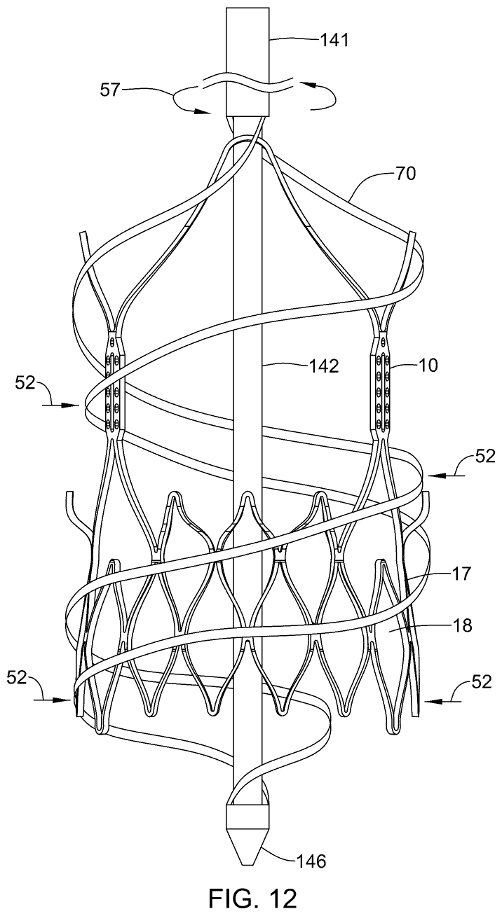

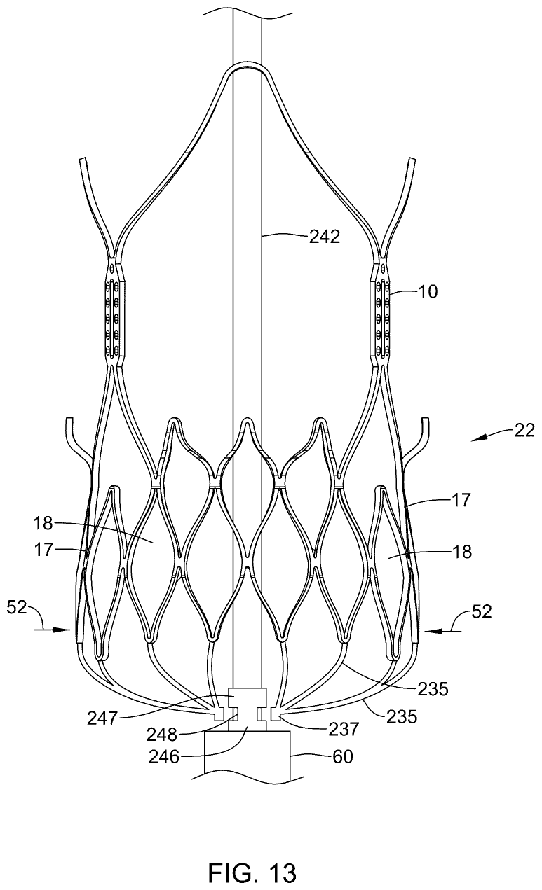

FIG. 13 illustrates an embodiment in which the lower portion 22 of the stent-valve 10 includes a plurality of struts 17 forming a plurality of open cells 18, and at least some of the plurality of struts 17 include distal extensions 235 that converge into at least two paddles 237. The cinching member includes a capture member 247 defined by the distal tip 246 fixed to the distal end of the inner shaft 242. The capture member 247 may be configured to releasably engage the at least two paddles 231. In some embodiments, the capture member 247 may include a plurality of recesses 248 each configured to releasably engage one of the paddles 231. The paddles 231 may be captured by the recesses 248 by rotation of the inner shaft 242 relative to the stent-valve 10. When the paddles 231 are received within the recesses 248, the distal sheath 60 may be moved proximally to radially compress the distal extensions 235 and lower portion 22 of the stent-valve 10, as indicated by arrows 52, and allow at least the distal end of the lower portion 22 of the stent-valve 10 to be re-inserted into the distal sheath 60. With the lower portion 22 of the stent-valve 10 compressed within the distal sheath 60, the stent-valve is effectively recaptured and may be moved axially for redeployment in a more desirable position.

In the embodiment shown in FIG. 14, the lower portion 22 of the stent-valve 10 includes a plurality of struts 17 forming a plurality of open cells 18, and the cinching member includes a plurality of recapture members 330 with first ends 331 fixed to the distal tip 346 fixed to the distal end of the inner shaft 342. The plurality of recapture members 330 may each have a free second end defining a hook 332 configured to releasably engage a lower series of open cells 18. When the hooks 332 are engaged with the open cells 18, the distal sheath 60 may be moved proximally to radially compress the recapture members 330 and the lower end 22 of the stent-valve 10, as indicated by arrows 52, and allow at least the distal end of the lower portion 22 of the stent-valve 10 to be re-inserted into the distal sheath 60. With the lower portion 22 of the stent-valve 10 compressed within the distal sheath 60, the stent-valve is effectively recaptured and may be moved axially for redeployment in a more desirable position. The recapture members 330 may be biased in a radially outward configuration in which, such that once the stent-valve has been deployed in a desired positioned, the inner shaft 342 may be moved proximally until the hooks 332 disengage from the open cells 18. The inner shaft 342 may then be moved distally to contract the recapture members 330 within the distal sheath 60. The entire delivery system may then be removed proximally through the stent-valve 10.

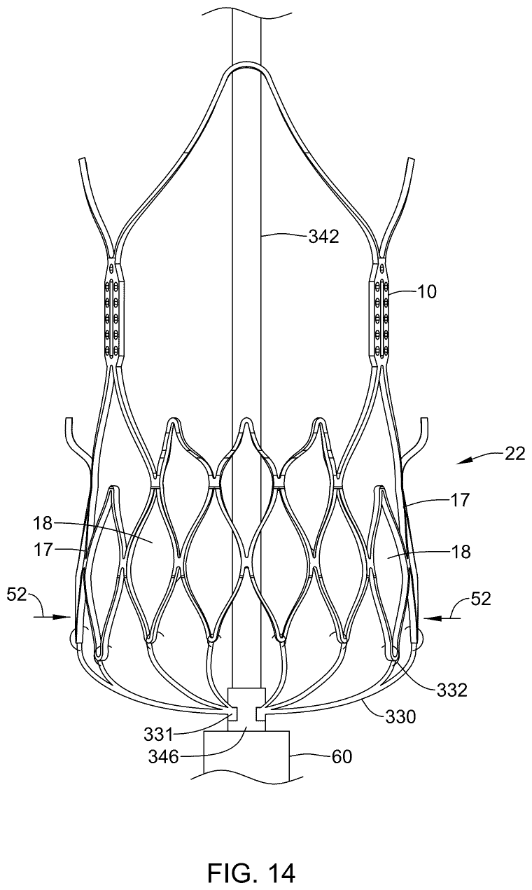

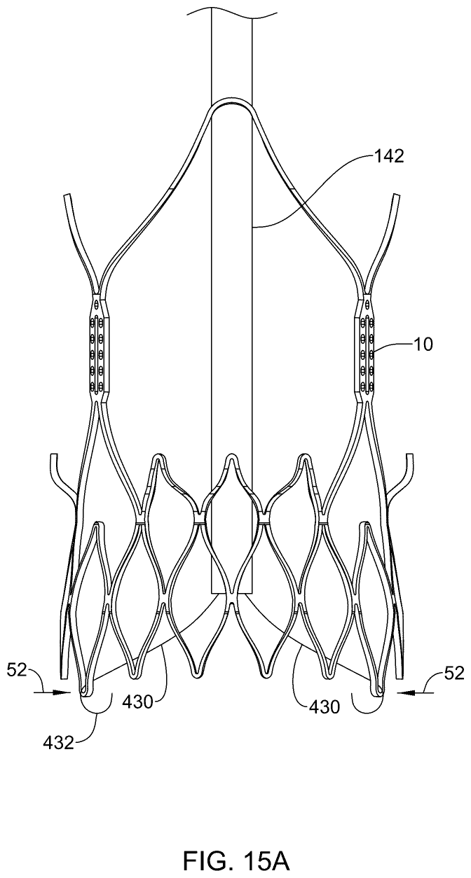

FIGS. 15A and 15B illustrate another recapture embodiment in which the at least one cinching member includes a plurality of shape memory hooks 430 slidably extending from the distal end of the inner shaft 142. Each of the hooks 430 may be configured to engage the cells defining the distal end of the stent-valve, as shown in FIG. 15A. When pulled proximally into the inner shaft 142, the hooks 430 may pull the distal end of the stent-valve 10 into a radially contracted configuration, as indicated by arrows 52. The hooks 430 may each be heat set with their free end 432 bent proximally, such that when the hooks are moved distally out of the inner shaft 142, the hooks disengage from the stent-valve, as shown in FIG. 15B.

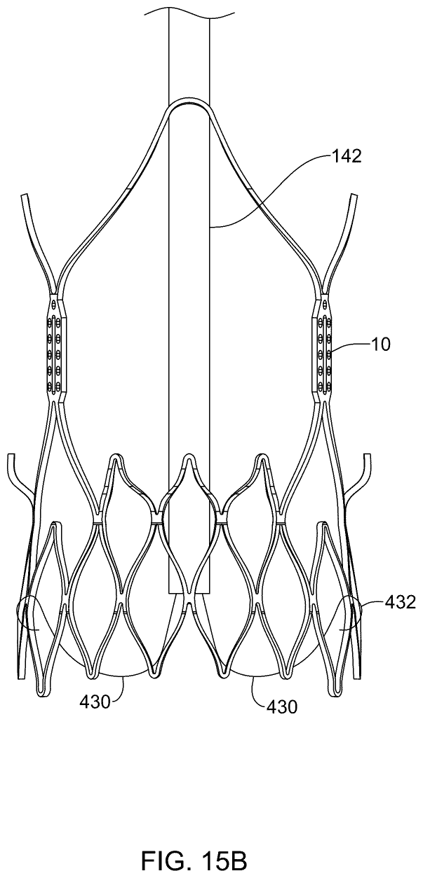

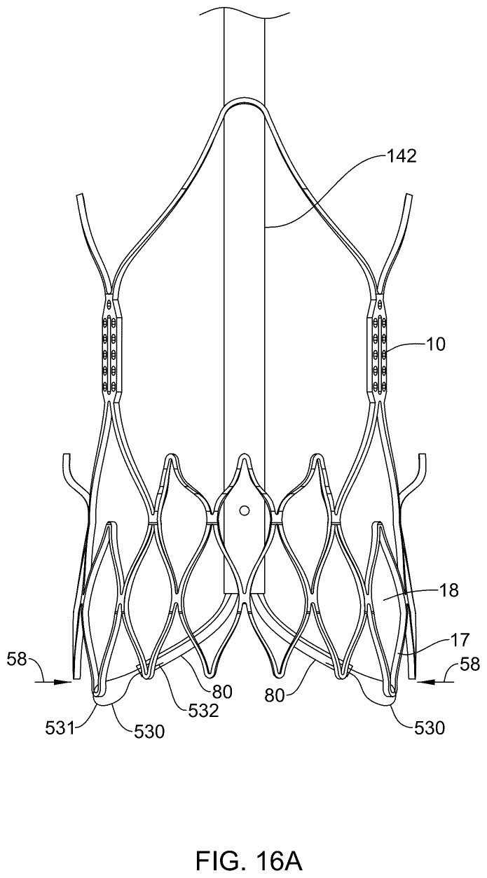

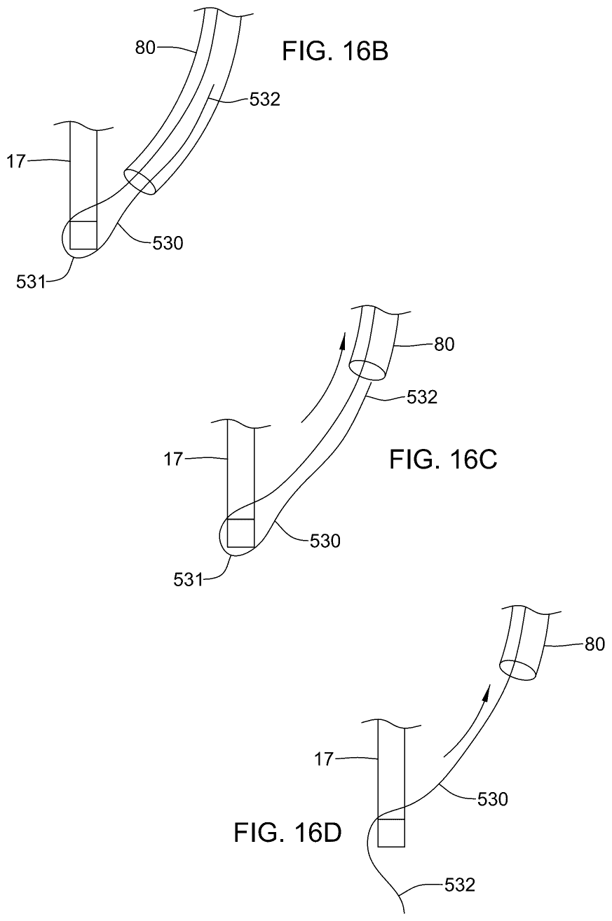

In another embodiment, the cinching member may include a plurality of release sheaths 80 slidably disposed within the inner shaft 142, where each release sheath 80 has a tether 530 slidably disposed therein, as shown in FIGS. 16A-16D. Each tether 530 may be formed from a relatively stiff wire or cable that has a relatively straight resting shape and resists bending such that when the tether 530 extends from the release sheath 80, wraps around one or two struts 17 of the stent-valve 10, and extends back into the release sheath 80, the release sheath 80 prevents the tether 530 from returning to its resting shape and secures the tether 530 to the strut 17. The tether 530 may wrap around one or two struts 17 by extending through an open cell 18. In some embodiments, the resting shape of the tether 530 may be straight or include a bend 531 proximal of the free distal end 532 of the tether. The bend 531 may be 90 degrees or less. For example, the bend 531 in the resting shape may be in the range of 1-90 degrees. In some examples, the tether 530 may be made from a shape memory material with the slight bend being the resting shape. During deployment of the stent-valve 10, the release sheaths 80 extend distally out of the inner shaft 142, with the tethers 530 forming a bend 531 as they wrap around the struts 17, with the bend 531 being approximately 180 degrees so the free distal end 532 may extend back into the release sheath 80. See FIGS. 16A and 16B. In this configuration, the release sheath 80 holds the distal end 532 of the tether 530 adjacent the body of the tether, thereby preventing the tether 530 from returning to its resting shape and releasing the strut 17. See FIG. 16B. The release sheaths 80 secure the tethers 530 to the struts 17, such that proximal movement of the tethers 530 pulls the distal end of the stent-valve 10 radially inward, as indicated by arrows 58. When the stent-valve 10 is in the final desired position, the release sheaths 80 may be pulled proximally relative to the tethers 530 until the distal ends 532 of the tethers 530 are released (FIG. 16C), at which point the tethers 530 return to their resting shape (FIG. 16D), releasing the strut 17 and allowing the tethers 530 to be pulled proximally from the stent-valve. See the progression of FIGS. 16B, 16C and 16D.

FIG. 17 illustrates another embodiment of recapture device in which the cinching member includes an expandable mesh 630 having a first end fixed to the distal tip 146 of the inner shaft 142 and a second free end 632 configured to extend over and surround the lower crowns 15 defining the distal end of the stent-valve 10. The expandable mesh 630 may be made of a shape memory material with the expanded configuration the at rest configuration. In the fully expanded configuration, the free end 632 of the expandable mesh 630 may have an inner diameter larger than the outer diameter of the distal end of the stent-valve 10. During deployment, the expandable mesh 630 is disposed over the lower crowns 15 of the stent-valve 10, allowing for a slower expansion of the distal portion of the stent-valve. In some embodiments, the expandable mesh 630 may include on or more hooks 633 configured to engage struts 17 of the stent-valve 10. After the distal sheath 60 has been withdrawn distally and the lower crowns 15 of the stent-valve 10 have expanded fully, the position of the stent-valve 10 may be checked for desired placement before disengaging the expandable mesh 630. If repositioning is desired, the distal sheath 60 may be moved proximally which causes the expandable mesh 630 to apply a radially inward force on the lower crowns 15, as indicated by arrows 59 to constrain the expandable mesh 630 and lower crowns 15 and allow for repositioning of the stent-valve 10. When the stent-valve 10 is in the desired position and the expandable mesh 630 has expanded fully, the inner shaft 142 may be moved proximately to disengage the hooks 633 from the struts 17. The inner shaft 142 may then be moved distally into the distal sheath 60 to collapse the expandable mesh 630 for removal from the body. If the expandable mesh 630 has been removed distally from the lower crowns 15 and then it is determined that further repositioning is desired, then with the distal sheath 60 positioned distal of the distal end of the expandable mesh 630, the inner shaft 142 may be moved proximally until the free end 632 is positioned proximal of the distal end of the stent-valve 10 and the hooks 633 if present, are engaged with struts 17, the distal sheath 60 may then be moved proximally to radially constrain the expandable mesh 630 and lower crowns 15, allowing for further repositioning of the stent-valve 10.

In some instances, a complete bail-out may desired, in which the fully expanded stent-valve 10 must be removed completely from the body. This bail-out procedure often needs to be performed rapidly to avoid injury to the patient. The upper anchoring crowns 13 in the stent-valve 10 shown in FIGS. 1 and 18A are designed to push the native leaflets out of the way and to prevent ventricular migration. However, the upper crowns 13 act as proximally facing hooks when attempting to pull the expanded stent-valve into a catheter 2 for bail-out. As shown in FIG. 18B, the upper crowns 13 may prevent the fully expanded stent-valve 10 from being pulled proximally into the catheter 2.

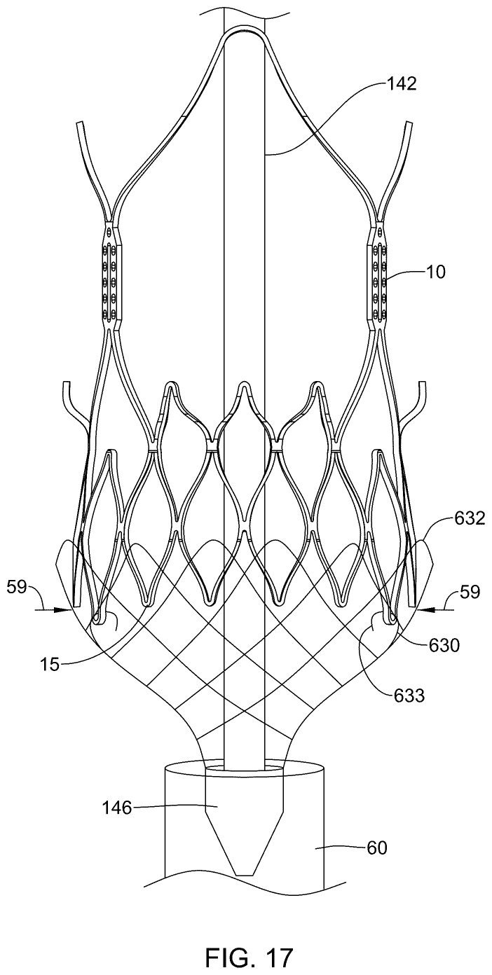

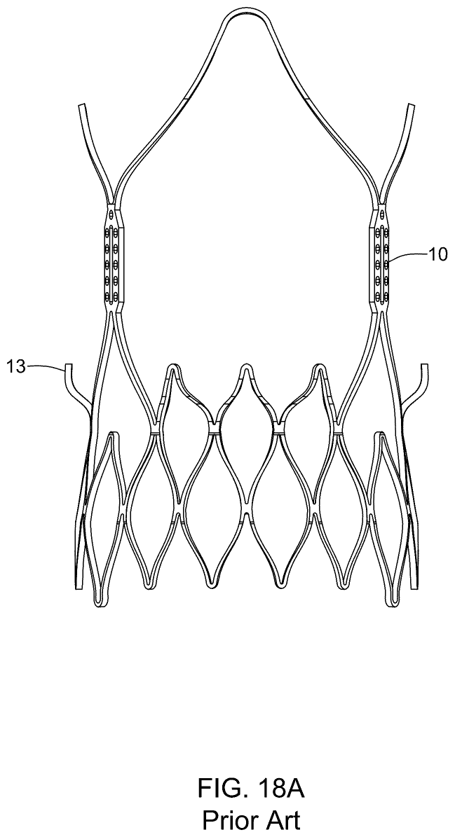

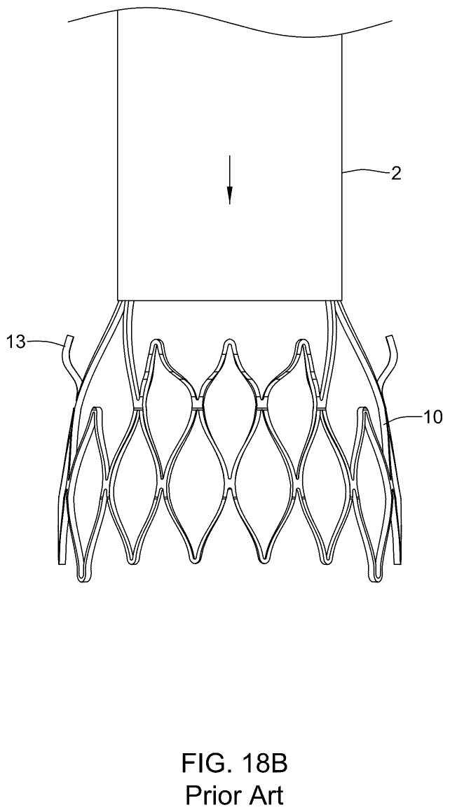

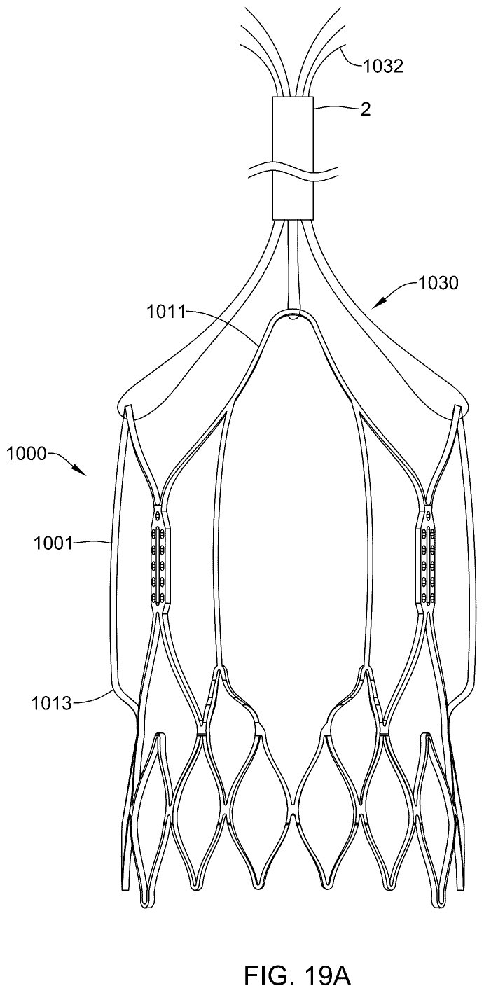

FIGS. 19A and 19B show different bail-out systems that each include a change to the upper anchoring crowns 1013 of the stent-valve 1000. The stent-valve 1000 includes an extension 1001 connecting each upper anchoring crown 1013 to one of the plurality of arches 1011. In this manner, the upper anchoring crowns 1013 form a radially extending curve instead of a hook. The bail-out system also includes at least one cinching member configured to releasably engage the plurality of arches 1011 and allow for the upper anchoring crowns 1013 to be pulled proximally into a catheter 2. In the embodiment shown in FIG. 19A, the cinching member includes a plurality of tethers 1030 such as sutures, each tether 1030 configured to extend distally from the catheter 2 and around one arch 1011. Free ends 1032 of the tethers 1030 may extend from a proximal end of the catheter 2 and may be pulled proximally to pull the arches 1011 into the distal end of the catheter. In the embodiment shown in FIG. 19B, the cinching member includes a plurality of couplers 1130 each having a distal end 1131 releasably coupled to an arch 1011 and a free end 1132 extending from the proximal end of the catheter 2. The distal end 1131 may include a hook or clamp configured to releasably engage the arch 1011. The tethers 1030 or couplers 1130 remain connected to the arches 1011 of the stent-valve 1000 during delivery and expansion of the stent-valve 1000. If bail-out is not needed, the tethers 1030 or couplers 1130 are disengaged from the arches 1011 and removed from the body.

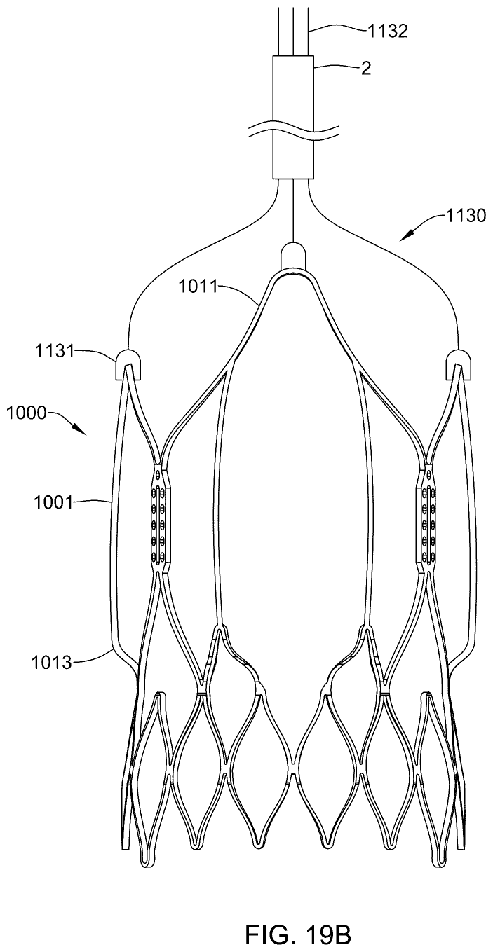

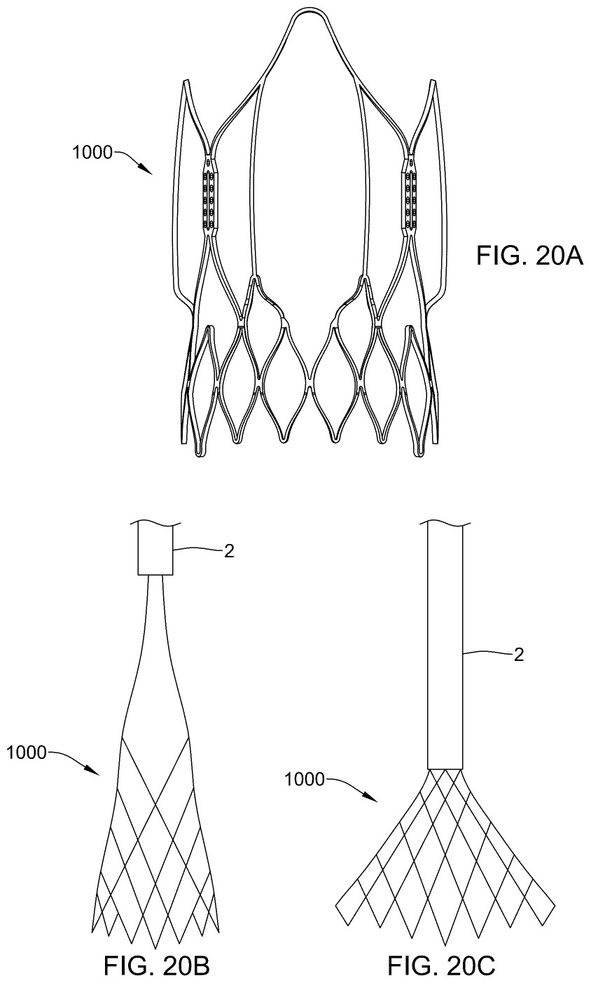

FIGS. 20A-20C are stepwise illustrations of a bail-out procedure of the stent-valve 1000 from FIGS. 19A-19B, demonstrating how the extensions 1001 connecting the upper anchoring crowns 1013 to the arches 1011 allow the stent-valve 1000 to be pulled proximally into the catheter 2. In some embodiments, an auxiliary device may be used to engage the stent-valve 1000 and pull it into the catheter 2, instead of having tethers or couplers already coupled to the arches, as described above.

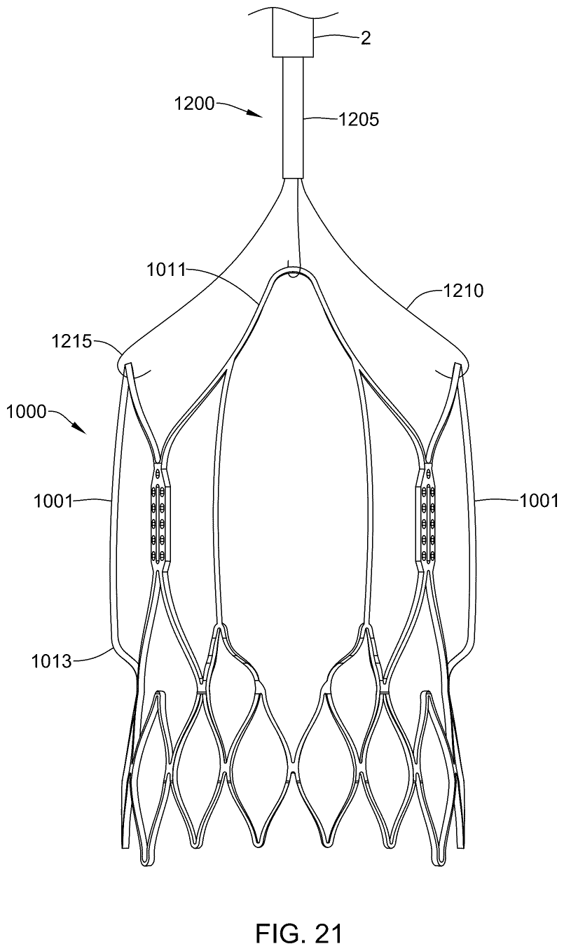

FIGS. 21-27B illustrate various structures that may be used in a bail-out procedure on the stent-valve 1000 with extensions 1001 connecting each upper anchoring crown 1013 to one of the plurality of arches 1011. FIG. 21 illustrates an extendable claw 1200 configured to be advanced through a catheter 2 to engage and pull the stent-valve 1000 proximally from the body. The extendable claw 1200 may include a shaft 1205 and a plurality of shape memory cables or wires 1210 slidably disposed within at least one lumen in the shaft 1205. Each wire 1210 may have an engagement member 1215 on a free distal end thereof. As shown in FIG. 21, the engagement members are hooks 1215 configured to engage the arches 1011 of the stent-valve 1000. The wires 1210 may be heat set in a radially expanded configuration such that when extended distally from the shaft 1205, the wires 1210 extend radially and the hooks 1215 may engage the arches 1011. The wires 1210 may then be pulled proximally into the shaft 1205 to radially compress the arches 1011, allowing the stent-valve 1000 to be pulled into the catheter 2 for removal from the body.

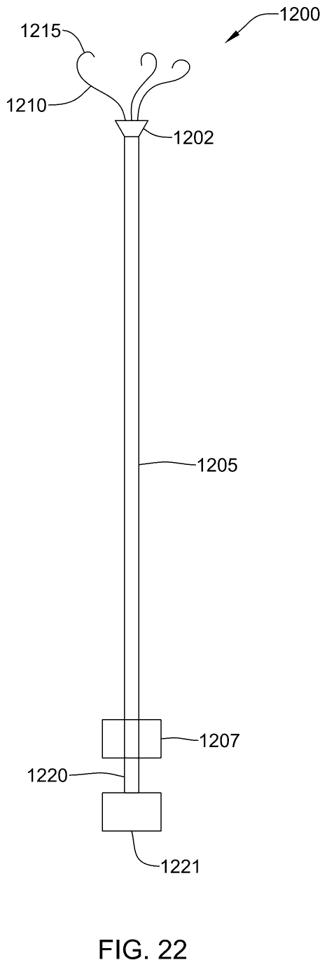

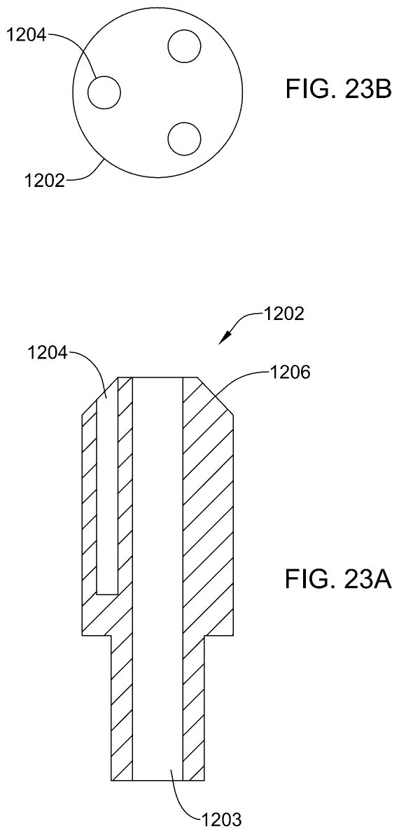

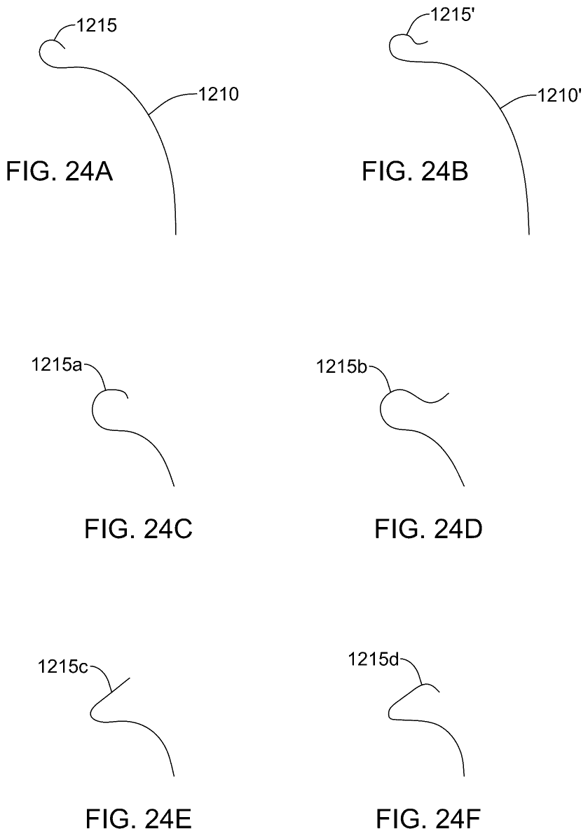

FIG. 22 illustrates an example claw device 1200, including an outer shaft 1205 with an outer handle 1207, and an inner shaft 1220 slidably disposed with the lumen of the outer shaft 1205 and having a proximal handle 1221. The plurality of wires 1210 may be fixed to a distal end of the inner shaft 1220, with each wire 1210 having a hook 1215 at the distal end thereof. In some embodiments, the plurality of wires 1210 may be fixed to a wire holder 1202 that is fixed to the distal end of the inner shaft 1220. FIGS. 23A and 23B illustrate an example wire holder 1202 that has a central lumen 1203 extending therethrough and is configured to receive a guidewire. The wire holder 1202 may have a plurality of axially extending channels 1204 each configured to receive an end of a wire 1210. In the example shown in FIGS. 23A and 23B, the wire holder 1202 has three channels 1204 spaced apart evenly around the distal face 1206 of the wire holder 1202. The distal face 1206 may include tapered surfaces through which the channels 1204 extend. The wire holder 1202 may separate and hold the wires 1210 apart and prevent them from becoming tangled when withdrawn into the outer shaft 1205. FIGS. 24C-24F illustrate various shapes of the hook 1215a-1215d on the distal end of the wires 1210. The hook 1215 may have a variety of shapes configured to enable the hook 1215 to easily engage and hold one of the arches 1011 of the stent-valve 1000.

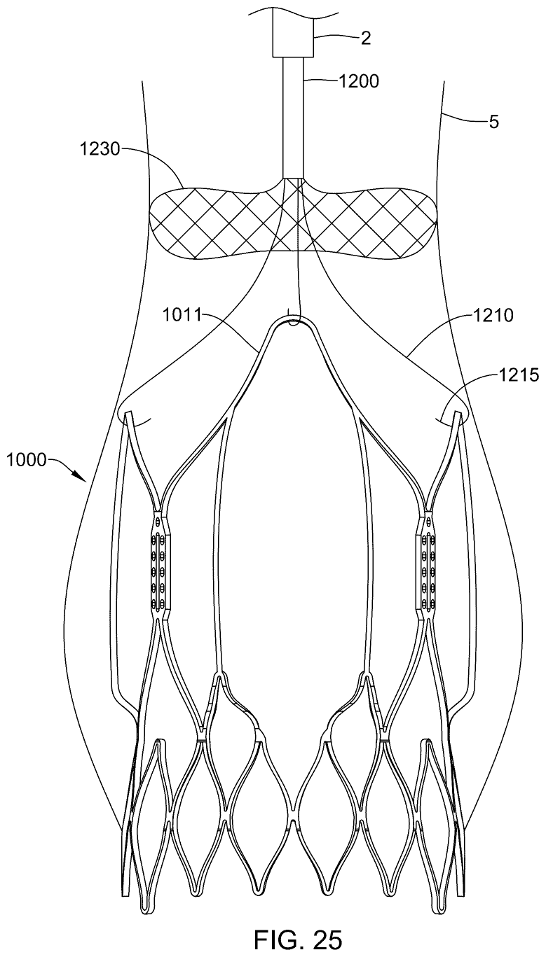

The delivery system may further include a centering member 1230 configured to center the wires 1210 as they extend out of the claw device 1200 extending distal from the catheter 2. as shown in FIG. 25, the centering member 1230 may include a shape memory braid or mesh shape set into a balloon or disc through which the wires 1210 extend. In some embodiments, the centering member 1230 may have a donut shape with an opening extending the center and through which the wires 1210 extend. In other embodiments, the wires 1210 may extend through openings in the braid or mesh. The centering member 1230 may be configured to expand radially and contact the inner walls of the aorta 5, thus centering the wires 1210 in the aorta as they extend distally from the claw device 1200, such that the hooks 1215 are positioned to engage the arches 1011 of the stent-valve 1000.

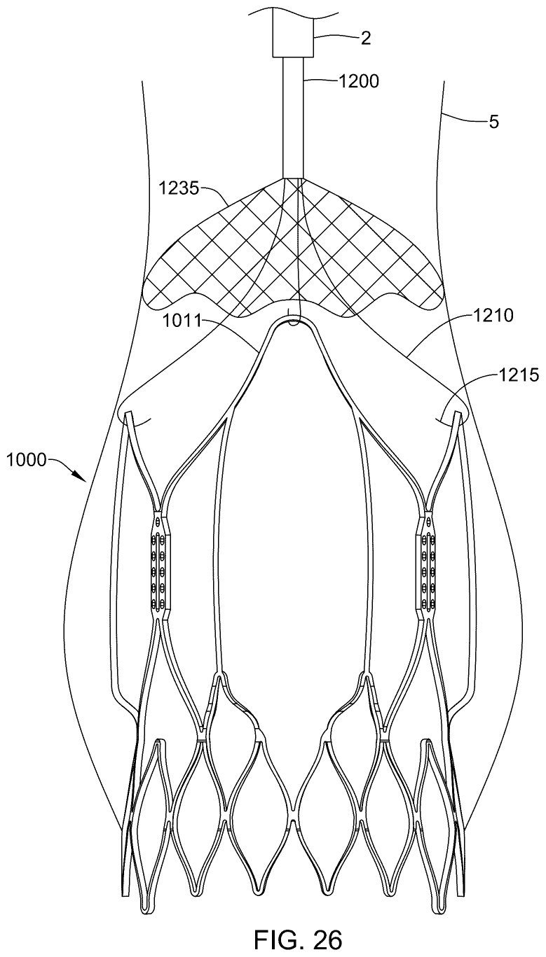

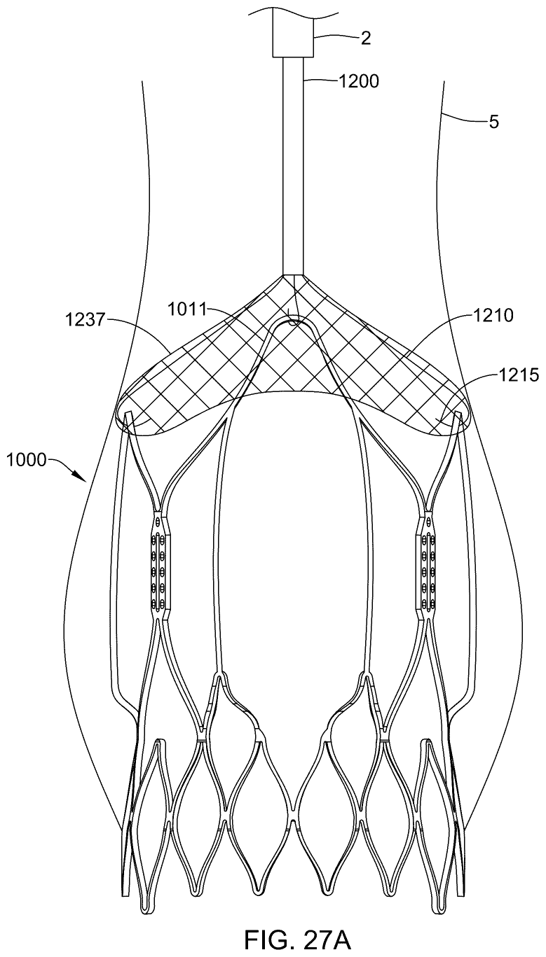

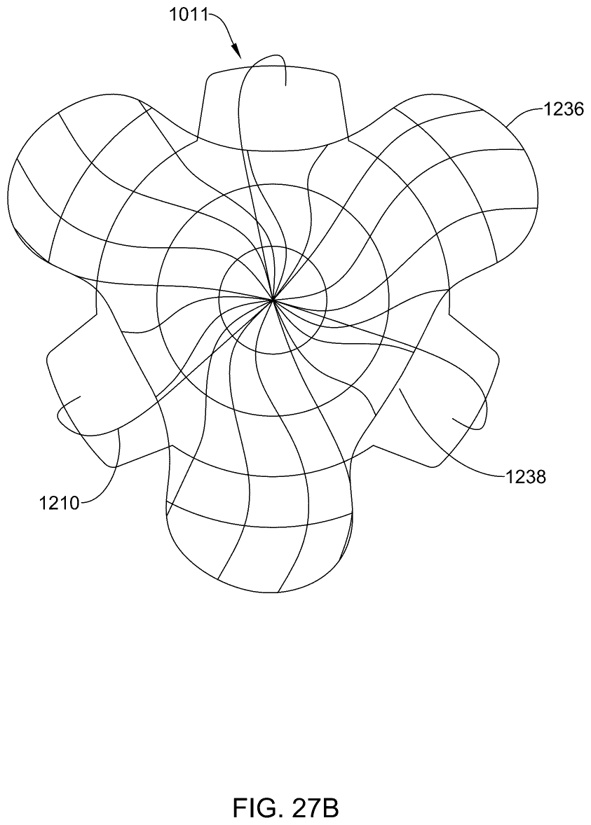

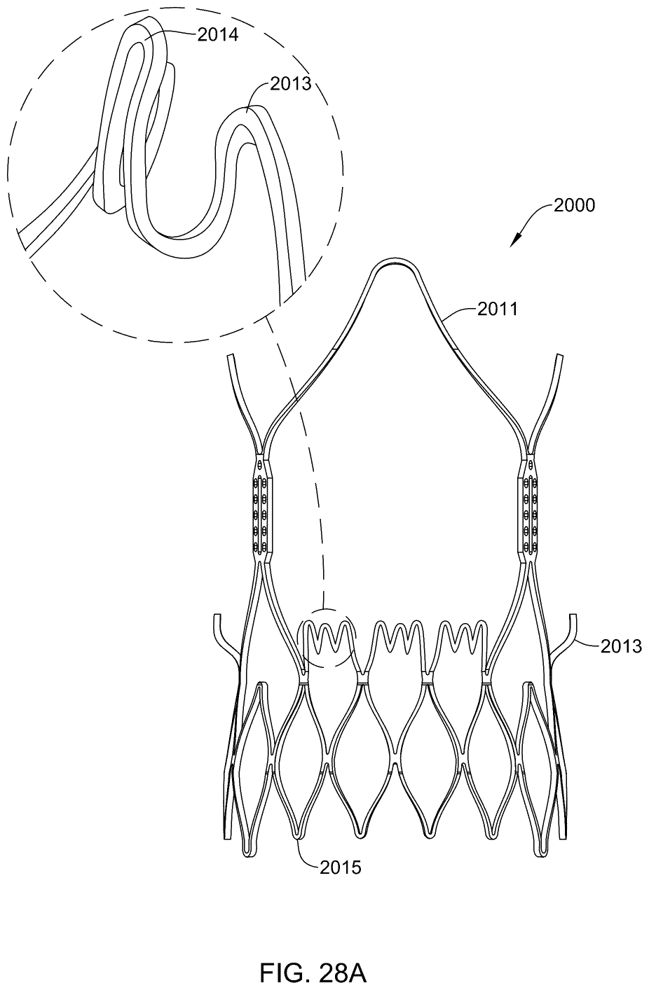

In another embodiment, the centering member is a braided or woven mesh cone 1235 configured to expand into contact with the inner walls of the aorta 5, as shown in FIG. 26. The wires 1210 may extend through openings in the braid or mesh as discussed above. FIGS. 27A and 27B illustrate a further embodiment in which the centering member is a triangular braided or woven structure 1237 with three radially extending lobes 1236 separated by depressions 1238 configured to receive one of the arches 1011 of the stent-valve 1000. The lobes 1236 may aid in positioning the wires 1210 into engagement with the arches 1011.

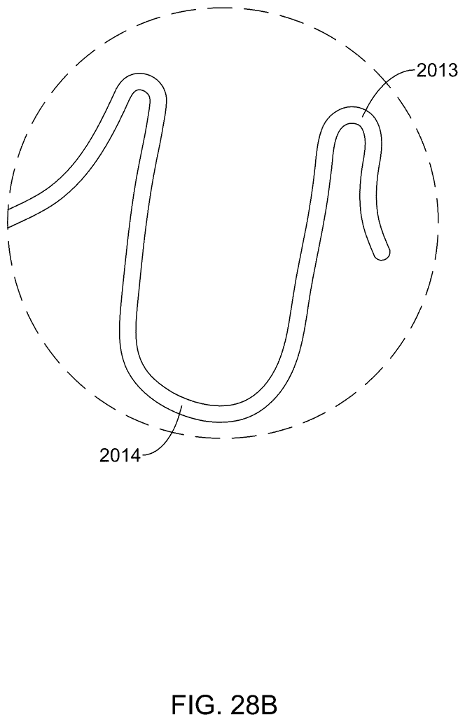

FIGS. 28A and 28B illustrate another embodiment of stent-valve 2000 in which the upper anchoring crowns 2013 are modified to allow for a bail-out procedure. The upper crowns 2013 have a J-shape configured to have a center extension 2014 extending proximally (toward the arches 2011) in a rest configuration, as shown in FIG. 28A and the enlarged region. The center extension 2014 is bendable such that when the stent-valve 2000 is pulled proximally into a catheter, the center extension 2014 bends distally (toward the lower crowns 2015) into a withdrawal configuration, as shown in FIG. 28B.

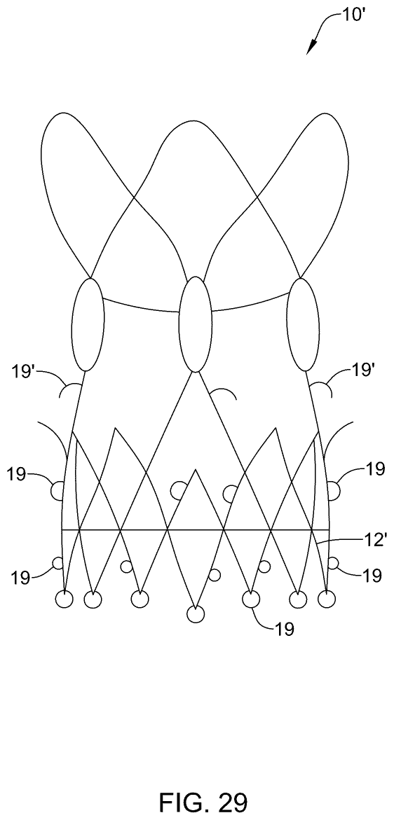

In addition to the various cinching and compressing members described above, the stent-valve may include one or more cinching member engagement elements configured to hold the cinching or compressing members in place. For example, as shown in FIG. 29, the stent-valve 10′ may include one or more engagement elements 19 positioned at one or more locations on the stent 12′. The engagement elements 19 may be loops, hooks, apertures, or other structures configured to receive the sutures, wires, and other cinching members described above. In some embodiments, the engagement elements 19 may be closed loops, through which the sutures may be threaded. In other embodiments, the engagement elements 19′ may be open loops, allowing the suture to be moved laterally into and out of the engagement elements.

The delivery system in accordance with any of the above described embodiments may be used in a method to deliver and reposition a replacement stent-valve. In some embodiments, the stent-valve may be used to replace the native aortic valve. In other embodiments, the stent-valve may be inserted into a failed replacement valve already implanted in the native aortic valve. The method of delivering and repositioning the stent-valve 10 may include inserting a distal tip of a stent-valve delivery system through a patient's aorta 5 and aortic valve 7, the delivery system including an inner shaft 142 including a distal tip 146, a stent-valve 10 crimped onto the inner shaft, the stent-valve including an upper portion 20, a lower portion 22, and a valve 14. The delivery system may further include a distal sheath 60 disposed over at least the lower portion of the stent-valve 10, a proximal sheath 40 disposed over at least the upper portion of the stent-valve 10, and at least one cinching member 30 coupled to at least the lower portion of the stent-valve 10 and extending through one of the inner shaft, the distal sheath, or the proximal sheath. The method further includes the steps of moving the proximal sheath 40 proximally to release the upper portion of the stent-valve, moving the distal sheath 60 distally to release the lower portion of the stent-valve, and cinching the cinching member 30 thereby radially compressing at least the lower portion of the stent-valve 10. The method may further include the steps of repositioning at least the lower portion of the stent-valve, and releasing the cinching member 30 thereby allowing the lower portion of the stent-valve to expand.

The materials that can be used for the various components of the delivery system (and/or other systems or components disclosed herein) and the various elements thereof disclosed herein may include those commonly associated with medical devices.

In some embodiments, delivery system (and variations, systems or components thereof disclosed herein) may be made from a metal, metal alloy, ceramics, zirconia, polymer (some examples of which are disclosed below), a metal-polymer composite, combinations thereof, and the like, or other suitable material. Some examples of suitable metals and metal alloys include stainless steel, such as 444V, 444L, and 314LV stainless steel; mild steel; nickel-titanium alloy such as linear-elastic and/or super-elastic nitinol; cobalt chromium alloys, titanium and its alloys, alumina, metals with diamond-like coatings (DLC) or titanium nitride coatings, other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R44035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R44003 such as ELGILOY®, PHYNOX®, and the like); platinum enriched stainless steel; titanium; platinum; palladium; gold; combinations thereof; and the like; or any other suitable material.

As alluded to herein, within the family of commercially available nickel-titanium or nitinol alloys, is a category designated “linear elastic” or “non-super-elastic” which, although may be similar in chemistry to conventional shape memory and super-elastic varieties, may exhibit distinct and useful mechanical properties. Linear elastic and/or non-super-elastic nitinol may be distinguished from super-elastic nitinol in that the linear elastic and/or non-super-elastic nitinol does not display a substantial “super-elastic plateau” or “flag region” in its stress/strain curve like super-elastic nitinol does. Instead, in the linear elastic and/or non-super-elastic nitinol, as recoverable strain increases, the stress continues to increase in a substantially linear, or a somewhat, but not necessarily entirely linear relationship until plastic deformation begins or at least in a relationship that is more linear than the super-elastic plateau and/or flag region that may be seen with super-elastic nitinol. Thus, for the purposes of this disclosure linear elastic and/or non-super-elastic nitinol may also be termed “substantially” linear elastic and/or non-super-elastic nitinol.

In some cases, linear elastic and/or non-super-elastic nitinol may also be distinguishable from super-elastic nitinol in that linear elastic and/or non-super-elastic nitinol may accept up to about 2-5% strain while remaining substantially elastic (e.g., before plastically deforming) whereas super-elastic nitinol may accept up to about 8% strain before plastically deforming. Both of these materials can be distinguished from other linear elastic materials such as stainless steel (that can also be distinguished based on its composition), which may accept only about 0.2 to 0.44 percent strain before plastically deforming.

In some embodiments, the linear elastic and/or non-super-elastic nickel-titanium alloy is an alloy that does not show any martensite/austenite phase changes that are detectable by differential scanning calorimetry (DSC) and dynamic metal thermal analysis (DMTA) analysis over a large temperature range. For example, in some embodiments, there may be no martensite/austenite phase changes detectable by DSC and DMTA analysis in the range of about −60 degrees Celsius (° C.) to about 120° C. in the linear elastic and/or non-super-elastic nickel-titanium alloy. The mechanical bending properties of such material may therefore be generally inert to the effect of temperature over this very broad range of temperature. In some embodiments, the mechanical bending properties of the linear elastic and/or non-super-elastic nickel-titanium alloy at ambient or room temperature are substantially the same as the mechanical properties at body temperature, for example, in that they do not display a super-elastic plateau and/or flag region. For example, across a broad temperature range, the linear elastic and/or non-super-elastic nickel-titanium alloy maintains its linear elastic and/or non-super-elastic characteristics and/or properties.

In some embodiments, the linear elastic and/or non-super-elastic nickel-titanium alloy may be in the range of about 50 to about 60 weight percent nickel, with the remainder being essentially titanium. In some embodiments, the composition is in the range of about 54 to about 57 weight percent nickel. One example of a suitable nickel-titanium alloy is FHP-NT alloy commercially available from Furukawa Techno Material Co. of Kanagawa, Japan. Other suitable materials may include ULTANIUM™ (available from Neo-Metrics) and GUM METAL™ (available from Toyota). In some other embodiments, a super-elastic alloy, for example a super-elastic nitinol can be used to achieve desired properties.

In at least some embodiments, portions or all of the delivery system (and variations, systems or components thereof disclosed herein) may also be doped with, made of, or otherwise include a radiopaque material. Radiopaque materials are understood to be materials capable of producing a relatively bright image on a fluoroscopy screen or another imaging technique during a medical procedure. This relatively bright image aids a user in determining the location of the delivery system (and variations, systems or components thereof disclosed herein). Some examples of radiopaque materials can include, but are not limited to, gold, platinum, palladium, tantalum, tungsten alloy, polymer material loaded with a radiopaque filler, and the like. Additionally, other radiopaque marker bands may also be incorporated into the design of the delivery system (and variations, systems or components thereof disclosed herein) to achieve the same result.

In some embodiments, portions of the delivery system (and variations, systems or components thereof disclosed herein), may be made from or include a polymer or other suitable material. Some examples of suitable polymers may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly (alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), Marlex® high-density polyethylene, Marlex® low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro (propyl vinyl ether) (PFA), ethylene vinyl alcohol, polyolefin, polystyrene, epoxy, polyvinylidene chloride (PVdC), poly (styrene-b-isobutylene-b-styrene) (for example, SIBS and/or SIBS 50A), polycarbonates, ionomers, polyurethane silicone copolymers (for example, Elast-Eon® from AorTech Biomaterials or ChronoSil® from AdvanSource Biomaterials), biocompatible polymers, other suitable materials, or mixtures, combinations, copolymers thereof, polymer/metal composites, and the like. In some embodiments, the sheath can be blended with a liquid crystal polymer (LCP). For example, the mixture can contain up to about 6 percent LCP.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The disclosure's scope is, of course, defined in the language in which the appended claims are expressed.

What is claimed is:

1. A delivery system configured to deliver, adjust, and/or recapture a stent-valve, the delivery system comprising:
an inner shaft having a distal end, a proximal end, and at least one lumen extending from the distal end to the proximal end;
an expandable stent-valve configured to move between a compressed configuration and an expanded configuration, the stent-valve coupled to the inner shaft in the compressed configuration, the stent-valve having an upper portion including a plurality of arches and a plurality of radially outwardly extending upper anchoring crowns, a lower portion, and a valve;
a distal sheath disposed over at least the lower portion of the stent-valve;
a proximal sheath disposed over at least the upper portion of the stent-valve; and
at least one cinching member coupled to the stent-valve and extending through one of the inner shaft, the distal sheath, or the proximal sheath;
wherein the upper portion of the stent-valve is configured to move to the expanded configuration when the proximal sheath is withdrawn and the lower portion is configured to move to the expanded configuration when the distal sheath is withdrawn;
wherein after at least a portion of the stent-valve has been moved to the expanded configuration, the at least one cinching member is configured to at least partially radially compress at least a portion of the stent-valve.

2. The delivery system of claim 1, wherein the at least one cinching member is at least one suture coupled to the stent-valve.

3. The delivery system of claim 2, wherein the suture has a middle region coupled to the stent-valve, and first and second free ends extending through at least one opening in the inner shaft and extending proximally through the inner shaft lumen and out the proximal end of the inner shaft, wherein pulling one or both free ends proximally causes the middle region to radially compress at least a portion of the stent-valve.

4. The delivery system of claim 3, wherein the lower portion of the stent-valve includes a plurality of struts forming a plurality of open cells, wherein the suture is coupled to at least two struts.

5. The delivery system of claim 4, wherein the suture is a first suture positioned distal of the plurality of upper anchoring crowns, the delivery system further comprising a second suture coupled to the stent-valve, wherein the second suture is spaced apart from the first suture.

6. The delivery system of claim 15, wherein the at least one opening in the inner shaft includes a first opening positioned proximal of the distal end of the inner shaft, wherein at least one free end of the first suture extends through the first opening in the inner shaft, wherein at least one free end of the second suture extends through the distal end of the inner shaft, allowing the first and second sutures to be pulled independently or simultaneously to compress the lower portion of the stent-valve.

7. The delivery system of claim 6, wherein the first and second sutures each extend around a circumference of the stent-valve.

8. The delivery system of claim 6, wherein each of the first and second sutures extend around struts on opposite sides of the stent-valve.

9. The delivery system of claim 3, wherein the suture includes first and second sutures each coupled to the stent-valve proximal of the upper crowns and extending proximally through the inner shaft lumen, the delivery system further comprising third and fourth sutures coupled to at least two points of the distal end of the stent-valve and extending proximally through the inner shaft lumen.

10. The delivery system of claim 2, further comprising an outer shaft slidably disposed over the inner shaft, wherein the at last one suture includes first and second sutures each having a first end fixed to the inner shaft at a position proximal of the distal end of the inner shaft, and a second end coupled to the lower portion of the stent-valve, wherein when the outer shaft slides distally over the inner shaft, the outer shaft pulls the sutures radially inward causing the stent-valve to be compressed towards the inner shaft.

11. The delivery system of claim 3, further comprising a rod extending through the inner shaft, wherein the first and second free ends are twisted around the rod, such that rotating the rod pulls the middle region of the suture toward and into the inner shaft, thereby radially compressing the stent-valve.

12. The delivery system of claim 3, wherein the at least one opening in the inner shaft includes an opening spaced proximally from the distal end of the inner shaft, wherein the first and second free ends of the suture are wrapped around an outer surface of the inner shaft proximally from the distal end and then extend into the opening, such that as the inner shaft is rotated, the suture wraps around the outer surface, shortening the middle region of the suture and pulling the stent-valve radially inward into the compressed configuration.

13. The delivery system of claim 2, wherein the inner shaft has a distal tip, wherein the at least one suture includes a plurality of sutures each having a first end fixed to the distal tip, each suture coupled to the stent-valve and then extending into an opening in the inner shaft, the opening spaced proximally from the distal tip.

14. The delivery system of claim 1, wherein the lower portion of the stent-valve includes a plurality of struts forming a plurality of open cells, wherein the inner shaft has a distal tip, wherein the at least one cinching member includes a plurality of ribbons each having a first end fixed to the distal tip, the plurality of ribbons extending proximally and woven through the plurality of open cells and extending proximally along the inner shaft to the proximal end.

15. The delivery system of claim 1, further including an outer shaft slidably extending over the inner shaft, wherein the inner shaft has a distal tip, wherein the at least one cinching member includes a plurality of ribbons each having a first end fixed to the distal tip and a second end coupled to the outer shaft, the plurality of ribbons extending from the distal tip proximally and helically around an outer surface of the stent-valve, wherein the outer shaft is rotatable in an opposite direction as the inner shaft, thereby cinching the stent-valve as the ribbons contract radially inward.

16. The delivery system of claim 1, wherein the lower portion of the stent-valve includes a plurality of struts forming a plurality of open cells including lower open cells defining lower crowns, wherein the inner shaft has a distal tip, wherein the at least one cinching member includes an expandable mesh having a first end fixed to the distal tip and a second free end configured to extend over and surround the lower crowns, wherein the distal sheath is configured to be moved distally to allow the mesh and lower portion of the stent-valve to expand, and to be moved proximally to radially constrain the mesh and distal crowns.

17. The delivery system of claim 1, wherein each of the plurality of upper anchoring crowns includes an extension connecting the upper anchoring crown to one of the plurality of arches, wherein the at least one cinching member includes a plurality of cinching members, each cinching member configured to extend distally from a delivery catheter and releasably engage one of the plurality of arches.

18. The delivery system of claim 17, wherein the plurality of cinching members are sutures, wherein first and second free ends of each suture extend proximally through the delivery catheter and a middle portion of each suture forms a loop around one of the plurality of arches.

19. A delivery system configured to deliver, adjust, and/or recapture a stent-valve, the delivery system comprising:
an inner shaft having a distal end, a proximal end, and at least one lumen extending from the distal end to the proximal end;
an expandable stent-valve configured to move between a compressed configuration and an expanded configuration, the stent-valve coupled to the inner shaft in the compressed configuration, the stent-valve having an upper portion including a plurality of arches and a plurality of radially outwardly extending upper anchoring crowns, a lower portion, and a valve, the lower portion including a plurality of struts defining a plurality of open cells;
a distal sheath disposed over at least the lower portion of the stent-valve;
a proximal sheath disposed over at least the upper portion of the stent-valve; and
at least one cinching member coupled to the stent-valve and extending through one of the inner shaft, the distal sheath, or the proximal sheath;
wherein the at least one cinching member is configured to gradually radially compress and release at least a portion of the stent-valve in the absence of the proximal and distal sheaths.

20. A method of delivering and repositioning a stent-valve, comprising:
inserting a distal tip of a stent-valve delivery system through a patient's aorta and aortic valve, the delivery system including:
an inner shaft including the distal tip;
a stent-valve crimped onto the inner shaft, the stent-valve including an upper portion, a lower portion, and a valve;
a distal sheath disposed over at least the lower portion of the stent-valve;
a proximal sheath disposed over at least the upper portion of the stent-valve; and
at least one cinching member coupled to at least the lower portion of the stent-valve and extending through one of the inner shaft, the distal sheath, or the proximal sheath;
moving the proximal sheath proximally to release the upper portion of the stent-valve;
moving the distal sheath distally to release the lower portion of the stent-valve;
cinching the cinching member thereby radially compressing at least the lower portion of the stent-valve;
repositioning at least the lower portion of the stent-valve; and
releasing the cinching member thereby allowing the lower portion of the stent-valve to expand.
